(19) United States
(12) Patent Application Publication
(10) Pub. No.: US 2007/0122440 A1
(43) Pub. Date: May 31, 2007

(54) Methods for producing nanoparticles

(76) Inventors: Christopher W. Macosko, Minneapolis, MN (US); Thomas R. Hoye, St. Paul, MN (US); Jessica Anacker, Cottage Grove, MN (US); Robert K. Prud 'homme, Lawrenceville, NJ (US)

Correspondence Address: MUETING, RAASCH & GEBHARDT, P.A., P.O. BOX 581415, MINNEAPOLIS, MN, 55458, US

(21) Appl. No.: 11/486,620

(22) Filed: Jul. 14, 2006

Related U.S. Application Data
(60) Provisional application No. 60/700,855, filed on Jul. 20, 2005.

(51) Int. Cl. A61K 9/14; A61K 38/13; B29B 9/00; A01N 25/00

(52) U.S. Cl. 424/405; 424/489; 264/5; 514/2; 514/11; 977/906

(57) ABSTRACT

The present invention relates to methods of preparing nanoparticles from reactively formed block and/or graft copolymers and nanoparticles derived therefrom

### CROSS-REFERENCE TO RELATED APPLICATION

This application claims the benefit of U.S. Provisional Patent Application Ser. No. 60/700,855, filed Jul. 20, 2005, which is incorporated herein by reference in its entirety.

### GOVERNMENT FUNDING

The present invention was made with government support under Grant No. DMR-0212302 awarded by the Materials Research Science and Engineering Center of the National Science Foundation. The Government may have certain rights in this invention.

### BACKGROUND

Nanoparticles can be used as micro-reactor vessels, to modify the flow properties of materials, or for delivery of pharmaceuticals, cosmetics, pigments, or agricultural agents. In addition, formation of nanoparticles with polymers has been used in various industries to modify the miscible and volatile properties of target molecules. For instance, nanoparticles can be used to create a drug delivery system for therapeutic agents that are hydrophobic in nature and cannot be administered either orally or through intravenous injection because they are not water-soluble. Such therapeutic agents can be incorporated into nanoparticles dispersed in an aqueous solution resulting in a composition that is suitable for injection, inhalation, or oral administration. In addition, the particles can be made small enough for filtration purification and to assure that the nanoparticles will not clog capillaries or alveoli. Also, smaller particles can enhance transfer rates to the body or foliage due to the associated increase in the surface area per volume.

The formation of nanoparticles has been achieved by various methods. Nanoparticles can be made by precipitating a molecule in a water-miscible solvent and then drying and pulverizing the precipitate to form nanoparticles. Similar techniques for preparing nanoparticles for pharmaceutical preparations include wet grinding or milling. Other methods include mixing low concentrations of polymers dissolved in a water-miscible solution with an aqueous phase to alter the local charge of the solvent and form a precipitate through conventional mixing techniques. Other methods include the mixing of copolymers in organic solution with an aqueous phase containing a colloid protective agent or a surfactant for reducing surface tension. Other methods of incorporating additive therapeutic agents into nanoparticles for drug delivery require that nanoparticles be treated with a liposome or surfactant before drug administration.

Typically, current methods for forming nanoparticles by precipitation demonstrate little or no control of particle size and show poor yields. Uncontrolled and unpredictable particle size is particularly disadvantageous in the formation of pigment, pharmaceutical, and agricultural products. Furthermore, large-scale production of nanoparticles using established methods can be quite costly due to the low concentration of polymer initially introduced into the process solvent prior to nanoparticle production. Finally, many production techniques such as milling or wet grinding introduce the possibility of contamination into the final product.

In addition, methods for forming nanoparticles with additive target molecules contained within the nanoparticle typically have been performed with additives at very low ratios compared with polymer and at low absolute concentration. Therefore, the fraction of additive target molecule per nanoparticle is minimal, and the cost of production is high. It is desirable to lower the ratio of polymer to additive target molecule, to increase the number of resulting nanoparticles that contain additive target molecule, to increase the amount of additive target molecules contained within the nanoparticles, and to reduce the amount of initial polymer needed to create these nanoparticles.

For the foregoing reasons, there is a need for a process of creating nanoparticles in which the size and size distribution of the resulting nanoparticle can be predicted and controlled, additives can be incorporated into the nanoparticle at a high yield, and the amount of polymer initially needed reduced. Furthermore, there is a long felt need for a process of producing nanoparticles at a high concentration and in which the nanoparticles produced can be harvested easily and with a high yield.

### SUMMARY OF THE INVENTION

The present invention is aimed at methods of producing nanoparticles.

In one embodiment, the present invention provides a method of producing nanoparticles, the method including: providing a first reactant mixture including a first solvent stream and one or more polymeric reactants, wherein the first solvent stream includes one or more solvents; providing a second reactant mixture including a second solvent stream and one or more polymeric reactants, wherein the second solvent stream includes one or more solvents; and combining the reactant mixtures and flash precipitating under conditions to react the polymeric reactants and form precipitated nanoparticles including a block and/or graft copolymer. The polymeric reactants are preferably chosen such that they are immiscible with each other and react to form nanoparticles. In one embodiment, the reactant mixture(s) may include a mixture of polymeric reactants. The method of the present invention may also include providing one or more additional solvent streams and/or one or more additional reactant mixtures.

Preferably, flash precipitation of the nanoparticles is performed in a centripetal mixer, a continuous flash mixer, or a batch flash mixer. In one embodiment, the mixing velocity has a Reynold's number of at least 100, more preferably, the Reynold's number is at least 500.

In one embodiment, at least one of the polymeric reactant mixtures (e.g., the first polymeric reactant mixture) includes a polymeric reactant selected from functionalized polystyrenes, functionalized polycaprolactones, and mixtures thereof. Preferably, the first polymeric reactant includes acid chloride groups, isocyanate groups, or mixtures thereof.

In another embodiment, at least one of the polymeric reactant mixtures (e.g., the second polymeric reactant mixture) includes a polymeric reactant selected from functionalized polyethylene glycols, polypropylene oxides, and combinations thereof (e.g., copolymer, mixtures. Preferably, the second polymeric reactant includes amine groups.

In yet another embodiment, the polymeric reactants have a molecular weight of at least 300 g/mole.

In one embodiment, the methods of the present invention further include providing one or more additive target molecules. Preferably, the present invention includes providing the one or more additive target molecules in one or more of the reactant mixtures. The present invention also includes providing the one or more additive target molecules in other ways, such as in one or more other solvent streams.

In another embodiment, the copolymer and the one or more additive target molecules are present in the nanoparticles in a ratio of at least 1:20 by weight, and in another embodiment in a ratio of no greater than 99:1 by weight.

In yet another embodiment, at least one additive target molecule is selected from the group consisting of pharmaceutical actives and pharmaceutical precursor compounds. In still yet another embodiment, at least one additive target molecule is selected from the group consisting of agricultural organic compounds, biocides, pesticides, herbicides, fungicides, insecticides, and combinations thereof (e.g., conjugates, mixtures). In one embodiment, at least one additive target molecule is selected from the group consisting of cosmetic products, dyes, reagents, salts, biological markers, biological imaging agents, ink pigments, magnetic particles, radiopaque materials, and combinations thereof (e.g., conjugates, mixtures).

In still yet another embodiment, the present invention further includes a step of removing the solvents from the resultant mixture including the nanoparticles. Preferably, the solvents are removed by a process selected from the group consisting of filtration, distillation, evaporation, expansion, spray drying, lyophilization, centrifugation, extraction, and combinations thereof.

In one embodiment, the present invention provides nanoparticles prepared by the above methods.

Definitions

As used herein, the term “additive target molecule” refers to an active material that produces a desired effect.

As used herein, the term “block and/or graft copolymer” refers to any copolymer in which like monomer units occur in relatively long sequences.

As used herein, the term “copolymer” refers to a mixed polymer, the product of polymerization of two or more monomers at the same time.

As used herein, the term “electrophilic group” refers to a group that accepts an electron pair from a molecule, with which it forms a covalent bond.

As used herein, the term “flash precipitation” refers to a process whereby reactant polymers and optional target additives are dissolved in solvent(s), and rapidly mixed under conditions effective to precipitate the coproduct polymer and optional additives into nanoparticles.

As used herein, the term “immiscible” refers to substances that will not mix with each other to form a single phase.

As used herein, the term “nanoparticle” refers to a microscopic particle whose size is measured in nanometers.

As used herein, the term “nanoprecipitation” refers to the process whereby nanoparticles precipitate from solution.

As used herein, the term “nucleophilic group” refers to a group that gives up electrons, or a share in electrons, to a molecule, with which it forms a covalent bond.

As used herein, the term “average particle size” refers to the average of the longest dimension of a particle (e.g., for spherical particles, this is the diameter).

As used herein, the term “polymeric reactant” refers to a polymer with one or more reactive functional groups.

As used herein, the term “reactant mixture” refers to a mixture of at least one solvent stream and at least one polymeric reactant.

As used herein, the term “Reynold's number (Re)” is defined as Re=ρdv/η, where ρ is the density, d is the jet diameter, v is the jet velocity and η is the viscosity of the solvent.

As used herein, the term “supplemental additive” refers to a substance added, for example, to at least one reactant mixture and/or solvent stream, that modifies the resultant properties of the nanoparticles.

As used herein, “a,” “an,” “the,” “at least one,” and “one or more” are used interchangeably. The terms “comprises” and variations thereof do not have a limiting meaning where these terms appear in the description and claims. Thus, for example, a reactant mixture that comprises “a” polymeric reactant can be interpreted to mean that the reactant mixture includes “one or more” polymeric reactants. Similarly, a functionalized polymer comprising “a” functional group can be interpreted to mean that the functionalized polymer includes “one or more” of the same or different functional groups.

The term “and/or” means one or all of the listed elements (e.g., block polymer, graft polymer, or both a block polymer and graft polymer).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a representation of the nanoprecipitation via in situ formation of block copolymer.

FIG. 2 is a representation of a continuous flash mixer.

FIG. 3 is a representation of a batch flash mixer.

FIG. 4 is a representation of a GENDIST curve (using the REPES fit converted into the intensity distribution of particle sizes) and cryogenic transmission electron microscopy (Cryo-TEM) image (illustrating particle shape in solution) for reactively formed polystyrene-b-polyethyleneoxide block copolymer (PS-b-PEG) in THF/water. The average particle size is 121 nm by REPES, the larger particles in the Cryo-TEM image are due to swelling of particles by acid functional PS. The line through the image is due to the carbon grid that the sample was placed on before freezing.

FIG. 5 is a representation of GPC curves of reactively formed polystyrene-b-polyethyleneoxide block copolymer (PS-b-PEG) with β-carotene: (a) 4.2% (PS—COCl+PEG-NH₂) inlet concentration and (b) 9.1% (PS—COCl+PEG-NH₂) inlet concentration. The solid curve is the UV detector output and the dashed curves illustrate deconvolution of peaks for reacted and unreacted polystyrene. Correcting for a PS functionality of 75%, the conversion to block copolymer is ˜68% at the end of mixing for both samples.

FIG. 6 is a representation of GPC plots of the reaction of PS—COCl and PEG-NH₂ in THF/water at the outlet of the mixer. With a flow rate of 77 ml/min, the residence time in the mixer is approximately 20 ms. Conversion at this time was 70% with correction for functionality of polystyrene. The solid curve is the UV detector output and the dashed curves illustrate deconvolution of peaks for reacted and unreacted polystyrene.

FIG. 7 is a representation of a GENDIST curve for 1.3 wt-% (PS—COCl+PEG-NH₂) and 2.6 wt-% β-carotene mixed with a flow rate of 77 mL/min. GENDIST curve using the REPES fit converted into the intensity distribution of particle sizes. The average particle size is 163 nm.

FIG. 8 is a representation of a GENDIST curve and Cryo-TEM image for 2.6 wt-% (PS—COCl+PEG-NH₂) and 2.6 wt-% β-carotene mixed at a flow rate of 77 mL/min: (a) GENDIST curve using the REPES fit converted into the intensity distribution of particle sizes and (b) Cryo-TEM image illustrating particle shape in solution. The average particle size is 170 nm.

FIG. 9 is a representation of a GENDIST curve and Cryo-TEM image for 4.2 wt-% (PS—COCl+PEG-NH₂) and 2.6 wt-% β-carotene mixed at a flow rate of 77 mL/min: (a) GENDIST curve using the REPES fit converted into the intensity distribution of particle sizes and (b) Cryo-TEM image illustrating particle shape in solution. The average particle size is 201 nm.

FIG. 10 is a representation of a GENDIST curve and Cryo-TEM image for 4.2 wt-% (PS—COCl+PEG-NH₂) and 2.6 wt-% β-carotene with a flow rate of 77 mL/min, run 4 in Table 4.2: (a) GENDIST curve using the REPES fit converted into the intensity distribution of particle size and (b) Cryo-TEM image illustrating particle shape. The average particle size is 148 nm.

FIG. 11 is a representation of GENDIST curves for 4.2 wt-% (PS—COCl+PEG-NH₂) and 2.6 wt-% β-carotene with a flow rate of 77 mL/min, runs 5 and 6 in Table 4.2. (a) GENDIST curve using the REPES fit converted into the intensity distribution of particle sizes run 5. The average particle size is 150 nm. (b) GENDIST Curve using the REPES fit converted into the intensity distribution of particle sizes run 6. The average particle size is 144 nm.

FIG. 12 is a representation of a GENDIST curve and Cryo-TEM image for 9.1 wt-% (PS—COCl+PEG-NH₂) and 2.6 wt-% β-carotene mixed at a flow rate of 77 mL/min: (a) GENDIST curve using the REPES fit converted into the intensity distribution of particle sizes and (b) Cryo-TEM image illustrating particle shape in solution. The average particle size is 177 nm. The holes in the Cryo-TEM image are due to beam damage that occurred during the measurements.

FIG. 13 is a representation of the effect of PS-b-PEG block copolymer concentration on intensity average particle size. The polymer concentrations studied have little or no effect on the size of particles formed with β-carotene concentration held constant.

FIG. 14 is a representation of a GENDIST curve for 4.2 wt-% (PS—COCl+PEG-NH₂) and 2.6 wt-% β-carotene mixed with a flow rate of 39 mL/min. GENDIST curve using the REPES fit converted into the intensity distribution of particle sizes. The average particle size is 162 nm.

FIG. 15 is a representation of a GENDIST curve for 4.2 wt-% (PS—COCl+PEG-NH₂) and 2.6 wt-% β-carotene mixed with a flow rate of 20 mL/min. GENDIST curve using the REPES fit converted into the intensity distribution of particle sizes. The average particle size is 185 nm. The average particle size increased by lowering the jet velocity further from 3.3 m/sec to 1.7 m/sec.

FIG. 16 is a representation of plot of average particle size as a function of jet velocity extrapolated to low velocity.

FIG. 17 is a representation of GENDIST curve using the REPES fit converted into the intensity distribution of particle sizes at a concentration of 4.2 wt-% block copolymer (PS—COCl+PEG-NH₂) and 1.3 wt-% β-carotene with a flow rate of 77 mL/min. The average particle size is 143 nm. There was no significant change in particle size or PDI by decreasing the concentration of β-carotene.

FIG. 18 is a representation of GENDIST curve using the REPES fit converted into the intensity distribution of particle sizes at a concentration of 4.2 wt-% block copolymer (PS—COCl+PEG-NH₂) and 5.2 wt-% β-carotene with a flow rate of 77 mL/min. The average particle size is 157 nm. There was no significant change in particle size or PDI by increasing the concentration of β-carotene. This result suggests the saturation limit of β-carotene in particles is 2.6 wt-%.

FIG. 19 is a representation of effect of β-carotene concentration on particle size. The limit for the in situ formation of PS-b-PEG particles appears to be approximately 3 wt-% β-carotene from this trend line.

FIG. 20 is a representation of Beer's Law calibration curve for β-carotene in THF measured at a wavelength of 485 nm, the wavelength of maximum absorbance for β-carotene in THF. The slope of the line is the product of the molar absorptivity and the path length of the sample cell.

FIG. 21 is a representation of GPC traces of (a) PCL-COCl and PEG-NH₂ mixture with TEA; (b) PCL-COCl and PEG-NH₂ mixture without TEA; (c) PEG-NH₂ control; and (d) PCL-COCl control using THF as liquid phase and a refractive index detector.

FIG. 22 is a representation of particle size distribution (a) in intensity; (b) in mass; (c) in number by DLS REPES; and (d) SEM picture, using CIJ mixing in situ formed PCL-b-PEG without β-carotene

FIG. 23 is a representation of particle size distribution (a) in intensity; (b) in mass; (c) in number by DLS REPES; and (d) SEM picture, using CIJ mixing in situ formed PCL-b-PEG with β-carotene.

### DETAILED DESCRIPTION OF ILLUSTRATIVE EMBODIMENTS

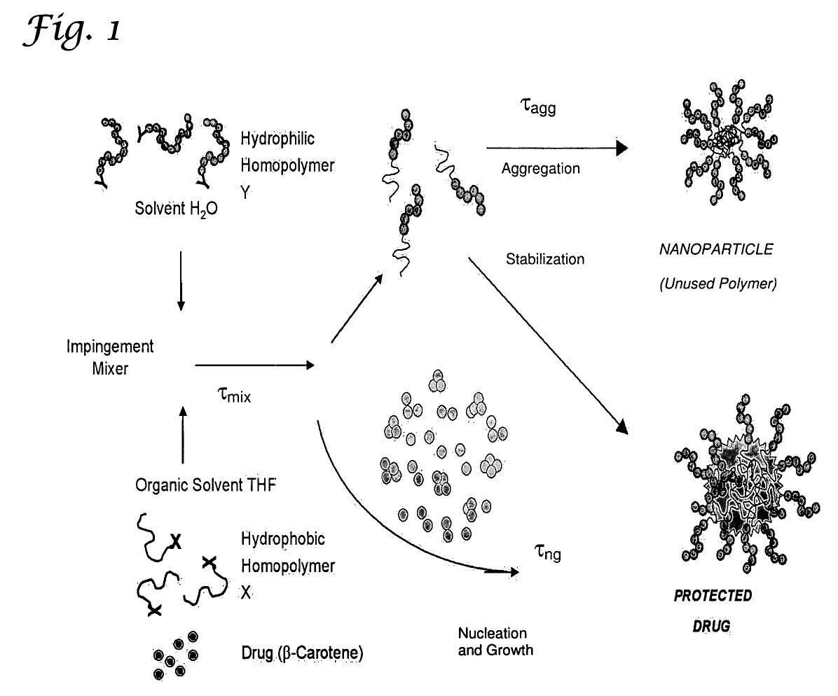

The present invention relates to methods of preparing nanoparticles from reactively formed block and/or graft copolymers and nanoparticles derived therefrom. The chemistry and the process conditions control the size of the resulting nanoparticles. The polymeric reactants are chosen such that they are generally immiscible and react to form a block and/or graft copolymer that can form nanoparticles. The first and second polymeric reactants are sufficiently dissolved in their respective solvents, rapidly mixed under conditions that form nanoparticles of block and/or graft copolymers, and the nanoparticles are flash precipitated under conditions described herein (see, e.g., FIG. 1).

This mixing can be achieved through various methods during which the mixing velocity is controlled. In addition, an additive target molecule can be mixed with the first and/or the second polymeric reactant and their respective solvents, or in other manners (e.g., in separate solvent stream(s) and/or other reactant mixtures) prior to flash precipitation for incorporation into the resulting nanoparticles.

Methods of the present invention may also include providing one or more additional solvent streams and/or one or more additional reactant mixtures. For example, in alternative embodiments, the method could include providing a third solvent stream, a fourth solvent stream, a third reactant mixture that includes one or more solvents and one or more polymeric reactants, a fourth reactant mixture that includes one or more solvents and one or more polymeric reactants, etc.

The present invention produces nanoparticles, for example, by flash precipitation using a centripetal mixer, a continuous flash mixer, or a batch flash mixer (see, e.g., Johnson et al., U.S. Patent Application Publication No. 2004/0091546). These mixing apparatuses are capable of reaching a critical and robust processing condition or a very fast mixing velocity and capable of controlling the size of the nanoparticles, by controlling mixing time (τₘ) through control of the mixing velocity. Mixing velocity is typically used to control the nanoparticle size distribution; however, quantifying the actual τₘ can be nontrivial. Therefore, it is practical to use the mixing velocity as an indicator of mixing time. For embodiments that use a continuous flash mixer, the mixing velocity is considered to be the highest average velocity of any of the fluids entering the mixing vessel. For embodiments that use a batch flash mixer, mixing velocity is considered to be the greater of either the moving surface velocity created by the tip speed or the average velocity of the incoming fluid. Actual mixing velocities may be, for example, higher or lower than the estimated mixing velocity of a single solvent stream or mix speed due to the cumulative effect of two fluids or moving surfaces coming together.

The mixing velocity of the reactant mixtures in a centripetal mixer, a continuous flash mixer, or a batch flash mixer, should be at least 0.02 meter per second (m/s).

The mixing velocity of the reactant mixtures in a centripetal mixer, a continuous flash mixer, or a batch flash mixer, should have a Reynold's number (Re) of at least 100, and preferably, at least 500. Reynold's number (Re) is based on the average of the inlet jet velocities.

The solvents are compositions that included of one or more fluid components and capable of carrying a solid or solids in solution or suspension. They are able to substantially dissolve polymeric reactants to a molecularly soluble state, for example, at a concentration of at least 0.001 wt-%. Solvents may be selected to be miscible or immiscible with each other.

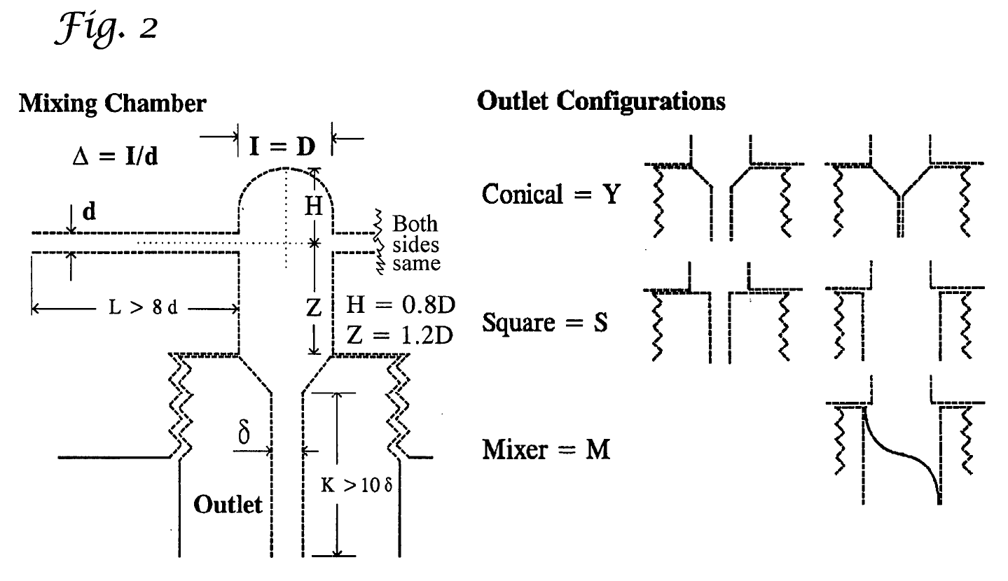

Methods of making nanoparticles, as described herein, may use, for example, a continuous flash mixer (see, e.g., FIG. 2, also called a confined impingement jet mixer, CIJ). In a preferred embodiment, two reactant mixture streams (including solvent streams and polymeric reactants) may be introduced into a mixing vessel through independent inlet tubes having a diameter, d, which can be about 0.25 millimeters (mm) to about 6 mm, but are about 0.5 mm to about 1.5 mm in diameter for laboratory scale production. The continuous flash mixer may include temperature control elements for fluid in the inlet tubes and in the mixing vessel.

Methods of making nanoparticles, as described herein, may use, for example, a continuous centripetal mixer. In the continuous centripetal mixer, the first reactant mixture stream (including, for example, a first solvent stream and a first polymeric reactant) and second reactant mixture stream (including, for example, a second solvent stream and a second polymeric reactant) may be directed into a mixing vessel and not directly impinge. The streams are typically forced to the walls of the mixing vessel by centripetal forces. In addition, the mixing vessel could be another high mixing velocity or highly confined mixer such as, but not limited to, a static mixer, rotor stator mixer, or a centripetal pump where the first and second reactant streams are introduced into the region of high mixing velocity. A variety of mixers capable of providing a sufficient mixing velocity with controlled introduction of the first and second reactant streams could afford a flash precipitation.

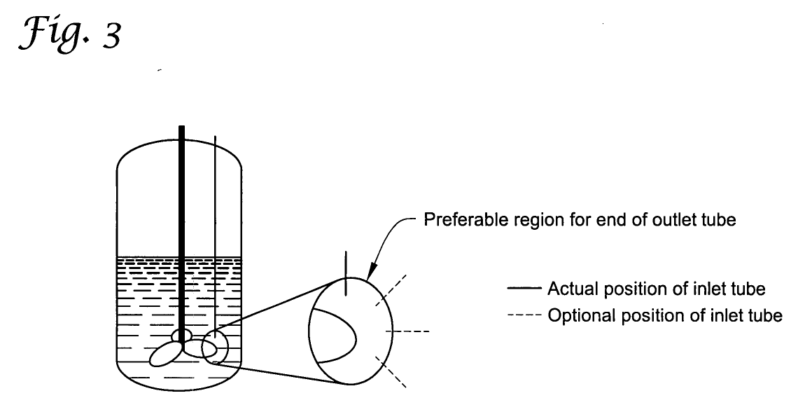

Methods of making nanoparticles, as described herein, may use, for example, a batch flash mixer (see, e.g., FIG. 3). In this method, a first reactant mixture stream, which contains a first polymeric reactant, a first solvent stream, and an optional additive target molecule, are added via an inlet tube to a second reactant mixture, which contains a second polymeric reactant, a second solvent stream, and either the same or a different optional additive target molecule, in a mixing vessel that has a mechanical agitator. The batch flash mixer includes temperature controlling elements for fluids in the inlet tubes and mixing vessel.

Formation of nanoparticles (and precipitation) of the block and/or graft copolymers are typically controlled by the selection of the polymeric reactants. Preferably, the polymeric reactants are chosen such that they are immiscible with each other. Precipitation of the block and/or graft copolymer upon mixing can also be effected by changes in temperature, composition, or pressure or any combination of each.

The nanoparticles, as disclosed herein, may have, for example, a polydispersity index (PDI) of less than 2, preferably, less than 1.2, and even more preferably, a polydispersity index of 1. The nanoparticles, as disclosed herein, may have, for example, an average particle size of less than 1000 nanometers (nm), preferably less than 300 nm, and even more preferably, less than 200 nm.

Suitable polymeric reactants include functionalized polymers (i.e., polymers with reactive functional groups). The functionalized polymers may include, for example, polymers prepared from the following subunits: acrylates including methyl acrylate, ethyl acrylate, propyl acrylate, n-butyl acrylate, isobutyl acrylate, 2-ethyl acrylate, and t-butyl acrylate; methacrylates including methyl methacrylate, ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; and lactic acids.

Preferred polymeric reactants may include, for example, functionalized polymers including polystyrenes, polycaprolactones, polyacrylates, polyethylene glycols, polypropylene oxides, polyglutamic acids, hyaluronic acids, polyvinylpyrrolidones, polylysines, polyspermines, polyarginines, alginic acids, polylactides, polyethylene imines, polyacrylic acids, polyiminocarboxylates, or combinations (e.g., copolymers, mixtures) thereof.

Such polymers are functionalized with reactive groups such as an acid chloride group, an isocyanate group, an anhydride group, an epoxide group, a ketone group, an aldehyde group, an enone group, enoate group, an alkyne group, another electrophilic group, or mixtures thereof. Such reactive groups are selected such that the polymeric reactants have complementary functional groups (i.e., groups that react with each other to form graft and/or block copolymers).

Preferably, the first polymeric reactant includes functionalized polystyrene, functionalized polycaprolactone, or mixtures thereof. The first polymeric reactant may include, for example, any reactive groups that will react with the functional groups of the second polymeric reactant. Preferably, the reactive groups of the first polymeric reactant include acid chlorides, isocyanates, or mixtures thereof.

Preferably, the second polymeric reactant includes functionalized polyethylene glycol, polypropylene oxide, or combinations (e.g., copolymers, mixtures) thereof. The second polymeric reactant may include, for example, an amino group, a hydroxyl group, a carboxylic acid group, an hydrazine group, an hydroxylamino group, a thiol group, an aromatic group, a heteroaromatic group, another nucleophilic group, or mixtures thereof. Preferably, the reactive groups of the second polymeric reactant include amines.

More than two different polymeric reactants can be used in the methods of the present invention if desired. Preferred methods, however, use two different polymeric reactants.

The polymeric reactants may have, for example, a concentration in each reactant mixture of at least 0.001 wt-%, preferably, at least 0.01 wt-%, more preferably, at least 0.1 wt-%, and even more preferably, at least 1 wt-%.

The polymeric reactants may have, for example, a molecular weight of at least 300 g/mole.

In one embodiment, the nanoparticles may include, for example, one or more additive target molecules in one or both of the reactant mixtures or in one or more solvent streams that do not include polymeric reactants.

The nanoparticles may include one or more additive target molecules at a suitable level to produce the desired result. In one embodiment, the block and/or graft copolymer and the one or more additive target molecules are preferably present in the nanoparticles in a ratio of at least 1:20 by weight. In another embodiment, the block and/or copolymer and the one or more additive target molecules are preferably present in the nanoparticles in a ratio of no greater than 99:1 by weight. In another embodiment, the block and/or copolymer and the one or more additive target molecules are preferably present in the nanoparticles in a ratio of no greater than 20:1 by weight.

Examples of some preferred additive target molecules that may be incorporated into the nanoparticles prepared by this method can be selected from the known classes of drugs including immunosuppressive agents such as cyclosporins (cyclosporin A), immunoactive agents, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immimosuppressants, antithyroid agents, antiviral agents, anxiolytic sedatives (hypnotics and neuroleptics), astringents, beta-adrenoceptor blocldng agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, anti-oxidants, preservatives, vitamins, nutrients, and combinations thereof (e.g., conjugates, mixtures). Preferred drug substances include those intended for oral administration and intravenous administration. They can be selected from any pharmaceutical organic active and precursor compound. A description of these classes of drugs and a listing of species within each class can be found in Physicians Desk Reference, 51^{st} edition, 2001, Medical Economics Co., Montvale, N.J., the disclosure of which is hereby incorporated by reference in its entirety. The drug substances are commercially available and/or can be prepared by techniques known in the art. Other additive target molecules may include, for example, agricultural compounds (e.g., biocides, pesticides, herbicides, fungicides, insecticides), peptides (e.g., proteins), cosmetic products, dyes, biological markers, ink pigments, magnetic particles, radiopaque materials, and combinations thereof (e.g., conjugates, mixtures).

In one embodiment, the additive target molecule is an organic active compound that is co-precipitated with a block and/or graft copolymer. The target molecule should be substantially insoluble in the mixture created after the mixing process is complete. The target molecule is typically supersaturated or above its solubility limit during the mixing process and precipitates in a characteristic time τ_{ng}. In a more preferred embodiment, the target molecule is poorly soluble in water (e.g., at a concentration of 1 wt-% or less, and more preferably, less than 0.1 wt-%) at a specific pH. In this case, the target molecule is preferably molecularly soluble in one of the reactant mixtures prior to the flash precipitation.

In one embodiment, the target molecule is an anti-oxidant or a pro-vitamin of poor water solubility (e.g., less than 0.1 wt-%). For instance, the target molecule may be β-carotene. The first polymeric reactant may include a reactive functionalized polystyrene. The second polymeric reactant may include a reactive functionalized polyethylene oxide (i.e., polyethylene glycol), reactive functionalized polypropylene oxide, or combinations (e.g., copolymers, mixtures) thereof. The first solvent is tetrahydrofuran and the second solvent is water. In this case, polyethylene oxide is presented to the outside of the molecule making the material dispersible in water.

In an exemplary embodiment, the mixture of first solvent stream containing the first polymeric reactant either alone or with an additive target molecule is mixed with a mixture of the second solvent stream containing the second polymeric reactant either alone or mixed with the same or an additional target molecule. At least one of the solvents is selected such that it is capable of changing the local molecular environment of the resulting block and/or graft copolymer and causing local precipitation of the block and/or graft copolymer.

Solvents can include water and typical organic solvents like acetic acid, acetic anhydride, acetone, acetonitrile, acrylonitrile, n-amyl alcohol, aniline, benzene, benzaldehyde, benzyl alcohol, n-butanol, butyl acetate, sec-butyl alcohol, chlorobenzene, chloroform, m-cresol, cyclohexane, cyclohexanone, dichloroethane, diethyl ether, diethylene glycol, N,N-dimethylacetamide, N,N-dimethylformamide, dimethylsulfoxide, 1,4-dioxane, ethanol, ethyl acetate, formic acid, n-hexane, methylene chloride, methyl ethyl ketone, N-methyl-2-pyrrolidone, nitrobenzene, nitromethane, phenol, n-propanol, ethylene glycol, propylene glycol, pyridine, sulfuric acid, tert-butyl methyl ether, tetrahydrofuran, toluene, triethanolamine, and trimethyl phosphate. At least one solvent is preferably water, which can be distilled, filtered, purified by reverse osmosis, or an aqueous solution containing a buffering agent, salt, colloid dispersant, and/or inert molecules.

The solvents can also be a mixture of solvents, such as alcohol and water.

Using the flash precipitation process described herein, nanoparticles are formed in the final mixture. The final solvent containing the nanoparticles can be altered by a number of post treatment processes, such as but not limited to dialysis, distillation, wiped film evaporation, centrifugation, lyophilization, filtration, sterile filtration, extraction, supercritical fluid extraction, spray drying, or combinations thereof. The processes typically occur after the nanoparticle formation but could also occur during the nanoparticle formation process.

One or more supplemental additives may be added to the first solvent or second solvent streams or to a stream of nanoparticles after formation by flash precipitation to tailor the resultant properties of the nanoparticles or for use in a particular indication.

The nanoparticles may include one or more supplemental additives at a suitable level to produce the desired result. The copolymer and the one or more supplemental additives are present in the nanoparticles in a ratio of at least 1:1000 by weight. In a preferred embodiment, the copolymer and the one or more supplemental additives are present in the nanoparticles in a ratio of no greater than 1000:1 by weight.

Suitable supplemental additives may include, for example, inert diluents, solubilizing agents, suspending agents, adjuvants, wetting agents, sweetening, flavoring, and perfuming agents, isotonic agents, colloidal dispersants and surfactants such as but not limited to a charged phospholipid such as dimyristoyl phophatidyl glycerol; alginic acid, alignates, acacia, gum acacia, 1,3-butyleneglycol, benzalkonium chloride, collodial silicon dioxide, cetostearyl alcohol, cetomacrogol emulsifying wax, casein, calcium stearate, cetyl pyridiniumn chloride, cetyl alcohol, cholesterol, calcium carbonate, CRODESTAS F-110, which is a mixture of sucrose stearate and sucrose distearate (Croda Inc.), clays, kaolin and bentonite, derivatives of cellulose and their salts such as hydroxypropyl methylcellulose (HPMC), carboxymethylcellose sodium, carboxymethylcellulose and its salts, hydroxypropyl celluloses, methylcellulose, hydroxyethylcellulose, hydroxypropylcellulose, hydroxypropylmethylcellulose phthalate, noncrystalline cellulose; dicalcium phosphate, dodecyl trimethyl aminonium bromide, dextran, dialkylesters of sodium sulfosuccinic (e.g., AEROSEL OT, American Cyanamid), gelatin, glycerol, glycerol monostearate, glucose, p-isononylphenoxypoly(glycidol), also known as OLIN 10-G or surfactant 10-GR (Olin Chemicals, Stamford, Conn.); glucamides such as octanoyl-N-methylglucamide, decanoyl-N-methylglucamide; heptanoyl-N-methylglucamide, lactose, lecithi(phosphatides), maltosides such as n-dodecyl-β-D-maltoside; mannitol, magnesium stearate, magnesium aluminum silicate, oils such as cotton seed oil, corn germ oil, olive oil, castor oil, and sesame oil; paraffin, potato starch, polyethylene glycols (e.g., CARBOWAX 3350 and CARBOWAX 1450, and CARBOPOL 9340 (Union Carbide)), polyoxyethylene alkyl ethers (e.g., macrogol ethers, such as CETOMACROGOL 1000), polyoxyethylene sorbitan fatty acid esters (e.g., TWEENS, ICI Specialty Chemicals), polyoxyethylene castor oil derivatives, polyoxyethylene sterates, polyvinylalcohol (PVA), polyvinylpyrrolidone (PVP), phosphates, 4-(1,1,3,3-tetramethylbutyl)-phenol polymer with ethylene oxide and formaldehyde, (also known ASTYLOXAPOL, SUPERIONE, and TRITON), poloxamers and polaxamines (e.g., PLURONICS F68LF, F87, F108 and TETRONIC 908 available from BASF Corporation, Mount Olive, N.J.), pyranosides such as n-hexyl-β-D-glucopyranoside, n-heptyl-β-D-glucopyranoside; n-octyl-β-D-glucopyranoside, n-decyl-β-D-glucopyranoside; n-decyl-β-D-maltopyranoside; n-dodecyl-β-D-glucopyranoside; quaternary ammonium compounds, silicic acid, sodium citrate, starches, sorbitan esters, sodium carbonate, solid polyethylene glycols, sodium dodecyl sulfate, sodium lauryl sulfate (e.g., DUPONAL P, DuPont), steric acid, sucrose, tapioca starch, talc, thioglucosides such as n-heptyl-β-D-thioglucoside, tragacanth, triethanolamine, TRITON X-200 (Rohm and Haas); and the like. The inert diluents, solubilizing agents, emulsifiers, adjuvants, wetting agents, isotonic agents, colloidal dispersants and surfactants are commercially available or can be prepared by techniques known in the art. The properties of many of these and other pharmaceutical excipients suitable for addition to the first and/or second solvent streams before or after mixing are provided in Handbook of Pharmaceutical Excipients, 3rd edition, editor Arthur H. Kibbe, 2000, American Pharmaceutical Association, London, the disclosure of which is hereby incorporated by reference in its entirety.

Colloidal dispersants or surfactants can be added to colloidal mixtures such as a solution containing nanoparticles to prevent aggregation of the particles. In one embodiment of the invention, a colloidal dispersant is added to either the first solvent or second solvent prior to mixing. In one embodiment, the colloidal dispersant can include a gelatin, phospholipid or pluronic. The dispersant is typically added in a ratio up to 2:1 with the additive target molecule by weight. The use of a colloidal dispersant can prevent nanoparticles from growing to a size that makes them undesirable.

In another embodiment of the invention, the additive target molecule is mixed with the first polymeric reactant with a supplemental seeding molecule. The inclusion of a supplemental seed molecule in the first solvent facilitates the creation of nanoparticles upon micromixing with the second solvent. Examples of a supplemental seed molecule may include, for example, but are not limited to, a substantially insoluble solid particle, a salt, a functional surface modifier, a protein, a sugar, a fatty acid, an organic or inorganic pharmaceutical excipient, a pharmaceutically acceptable carrier, or a low molecular weight oligomer.

The present invention is illustrated by the following examples. It is to be understood that the particular examples, materials, amounts, and procedures are to be interpreted broadly in accordance with the scope and spirit of the invention as set forth herein.

### EXAMPLES

The following examples are given to illustrate, but not limit, the scope of this invention. Unless otherwise indicated, all parts and percentages are by weight and all molecular weights are number average molecular weight. Unless otherwise specified, all chemicals used are commercially available from, for example, Sigma-Aldrich Corporation (St. Louis, Mo.).

ABBREVIATIONS, DESCRIPTIONS,
AND SOURCES OF MATERIALS

| | |
|---|---|
| | Acetonitrile (Anhydrous) |
| | Sigma-Aldrich Corporation |
| | β-Carotene (>97%) |
| | BASF Corporation |
| CDCl₃ | Deuterated Chloroform |
| | Sigma-Aldrich Corporation |
| DCM | Dichloromethane |
| | Sigma-Aldrich Corporation |
| HDI | Hexamethylene Diisocyanate |
| | Sigma-Aldrich Corporation |
| | Oxalyl Chloride |
| | Sigma-Aldrich Corporation |
| PB—NH₂ | Polybutadiene with Amine |
| | Functionality (PB—NH₂) |
| | Polymer Source Corporation |
| PEG-NH₂ | PEG with Amine End Functionality |
| | Nektar, Inc. |
| | Phenyl Isocyanate |
| | Sigma-Aldrich Corporation |
| PS—COCl | Polystryene with Acid Chloride |
| | End Functionality |
| | Polymer Source Corporation |
| PS—COOH | Polystyrene with Carboxyl |
| | End Functionality |
| | Polymer Source Corporation |
| PS-b-PEG | Polystyrene-b-poly(ethylene glycol) |
| PS—OH | Polystyrene with Hydroxyl |
| | End Functionality, Mn = 6000 g/mol |
| | Polymer Source Corporation |
| | Propylamine |
| | Sigma-Aldrich Corporation |
| TEA | Triethylamine |
| | Sigma-Aldrich Corporation |
| THF | Tetrahydrofuran (HPLC) |
| | Sigma-Aldrich Corporation |
| | Tin(II) Octanoate |
| | Sigma-Aldrich Corporation |
| | Toluene (HPLC) |
| | Sigma-Aldrich Corporation |
| | Water (HPLC) |
| | Sigma-Aldrich Corporation |
| CSA | Camphor Sulfonic Acid |
| | Sigma-Aldrich Corporation |
| | Capric Acid |
| | Sigma-Aldrich Corporation |
| | ε-Caprolactone |
| | Sigma-Aldrich Corporation |
| DMF | Dimethylformamide |
| | Sigma-Aldrich Corporation |
| PCL-COOH | Polycaprolactone with Carboxylic |
| | Acid End Functionality |
| PCL-COCl | Polycaprolactone with Acid |
| | Chloride End Functionality |

### Test Methods

### Gel Permeation Chromatography

Each sample was analyzed for block copolymer conversion using a Waters 717 Plus Autosampler connected to a Waters 590 programmable high pressure liquid chromatography (HPLC) pump running at a flow rate of 1 milliliter per minute (mL/min) at room temperature with THF as the mobile phase. Samples were capped with a 2 molar excess of phenyl isocyanate to prevent amine groups from interacting with the column. Block copolymer conversion was detected using a Spectroflow 727 ultraviolet (UV) absorbance detector set at a wavelength of 254 nanometers (nm), the wavelength for maximum PS absorption. Calibration was set using two sets of 5 polystyrene standards of known molecular weights ranging from 580 to 377400 grams per mole (g/mol).

### Confined Impingement Jet (CIJ) Mixing

CIJ mixing experiments were carried out using a mixer shown in FIG. 2. The volume of the mixer chamber is 21.2 mm³. Two mixer inlets were connected with two 100 mL gas tight glass syringes (SGE Inc.) via SWAGELOCK fittings and 1/16 inch inner (⅛ inch outer) diameter TEFLON tubing. The syringes were loaded on an infusion syringe pump (Harvard Apparatus model 975) capable of flow rates up to 77 mL/min with 100 mL syringes. The outlet of the mixer was connected with a five inch long and 1/16 inch inner (⅛ inch outer) diameter TEFLON tubing, which inducted the outlet fluid into a glass jar containing H₂O. The residence time of streams was estimated to be 113 milliseconds (ms), which corresponded to a stream travels from inlets of the mixer to the jar, if experiments was performed at a flow rate of 72 mL/min or jet velocity of 6.1 meters per second (m/s).

### Cryogenic Transmission Electron Microscopy (Cryo-TEM)

A controlled environment vitrification system (CEVS) was used in preparation of all samples before insertion into the cryogenic holder for transmission electron microscopy (TEM) measurements (see, e.g., Bellare et al., J. Electron Microsc. Tech. 1988, 10, 87-111). The sample was transferred to a Gatan 626 Single Tilt Cryo-Transfer System and images were taken using a JOEL 1210 transmission electron microscope cooled by liquid nitrogen operating at temperature of −170° C.

### Scanning Electron Microscopy (SEM)

SEM employs scattered electrons instead of light wave to create images. It can provide magnified images of fine objects. Each sample was first diluted by a factor of 10 with HPLC grade water, and then was sprayed on the surface of a cleaned silica wafer. After evaporation of solvent at room temperature, the sample was coated with a 30 Å layer of platinum by a sputter coater, and is loaded to a JEOL 6500 scanning electron microscope for measurement.

### Dynamic Light Scattering (DLS)

Samples were prepared by diluting solutions from mixing experiments by a factor of 10 with HPLC grade water to afford a concentration of approximately 0.02-0.04 weight percent (wt-%) block copolymer. Samples including polystyrene (PS) component were passed through a 0.42 micrometer (μm) filter (Millipore) into ¼ inch (in) diameter optical glass tubes, which had been cleaned with HPLC grade THF and dichloromethane and dried in a drying oven for 24 hours. Samples including polycaprolactone (PCL) component were directly filled into glass tubes without filtration.

Particle sizes were analyzed on a photometer equipped with an electrically heated silicon oil bath, Lexel 95-2 Ar⁺ laser operating at a wavelength of 488 nm, Brookhaven BI-DS photomultiplier, and Brookhaven BI-900 correlator (see, e.g., Lodge et al., Langmuir 2003, 19, 2103-2109). The intensity correlation function, g⁽²⁾(t), was collected and in situ calculated for each sample at a minimum of five angles between 60° and 120° with a Brookhaven BI-9000 correlator at 25° C. Collection time for each angle was approximately 12 minutes.

Correlation functions were analyzed using cumulant (see, e.g., Koppel, D. E. J. Chem. Phys. 1972, 57, 4814-4820), and REPES (see, e.g., Schillen, K.; Brown, W.; Johnsen, R. M. “Micellar sphere-to-rod transition in an aqueous triblock copolymer system. A dynamic light scattering study of translational and rotational diffusion”, Macromolecules 1994, 27, 4825-4832., Brown, W. Dynamic Light Scattering: The Method and Some Applications; Oxford Science Publications: New York, 1993, and Jakes, J. “REPES—a nonlinear regularization method for inverse Laplace transformation”, Collect. Czech. Chem. Comm. 1995, 60, 1791-1797) methods to determine an average particle size and distribution in intensity. GENDIST, a software package developed to generate the intensity distribution (G (Γ)) of decay rate (Γ), was used for the REPES algorithm. Mass and number average particle sizes and their distributions were estimated from the corresponding intensity ones. An intensity average particle size D̅_̅{̅I̅}̅ or Dₕ provided by DLS was defined as
D̅_̅{̅I̅}̅=ΣnᵢDᵢ⁶/ΣnᵢDᵢ⁵
where nᵢ was a number of particles with a diameter Dᵢ.
The corresponding intensity polydispersity index PDI_{I} was defined as

$${PDI}_{I} = \frac{\sum {n}_{i} {D}_{i}^{7} / \sum {n}_{i} {D}_{i}^{6}}{\sum {n}_{i} {D}_{i}^{6} / \sum {n}_{i} {D}_{i}^{5}}$$

A mass average particle size D̅_̅{̅w̅}̅ provided by DLS was defined as
D̅_̅{̅W̅}̅=ΣnᵢDᵢ/ΣnᵢDᵢ³
The corresponding mass polydispersity index PDI_{w} was defined as

$${PDI}_{w} = \frac{\sum {n}_{i} {D}_{i}^{5} / \sum {n}_{i} {D}_{i}^{4}}{\sum {n}_{i} {D}_{i}^{4} / \sum {n}_{i} {D}_{i}^{3}}$$

A number average particle size D̅ₙ̅ provided by DLS was defined as
D̅ₙ̅=ΣnᵢDᵢ/nᵢ
The corresponding number polydispersity index PDIₙ was defined as

$${PDI}_{n} = \frac{\sum {n}_{i} {D}_{i}^{2} / \sum {n}_{i} {D}_{i}}{\sum {n}_{i} {D}_{i} / \sum {n}_{i}}$$

### Particle Size Analysis

Particle sizes were measured at room temperature on a commercial particle size analyzer (LS230, Coulter) when the particles were known to be greater than 420 nm from dynamic light scattering sample preparation.

### Ultraviolet Visible (UV-vis) Spectrometry

Ultraviolet/Visible Spectrometry (UV-vis) was used to determine the percentage of β-carotene encapsulated by the PS-b-PEG nanoparticles formed by the in situ process. Samples were allowed to sediment by gravity after dialysis for at least 2 months in the dark before measurements were made. The supernatant was removed from the precipitate. The collected precipitate was allowed to dry for one week at room temperature in the dark, as β-carotene can oxidize when exposed to light. The precipitate was dissolved in 100 milliliter (mL) of THF for measurement. A series of calibration standards of β-carotene in THF were prepared with molar concentrations ranging from 0.22 millimolar per liter (mmol/L) to 9.8 mmol/L. Measurements of absorbance were carried out using a Hewlett Packard 8453 diode array spectrophotometer equipped with an Agilent 89090A temperature controller set at cell temperature of 25° C. An 1.75 mL glass microcuvette with 2 frosted sides and a path length of 1 centimeter (cm) was used for all measurements. All absorbance measurements were recorded at a wavelength of 485 nm, the maximum absorption for β-carotene in THF (see, e.g., Laughlin et al., Chem. Phys. Lipids, 2002, 115, 63-76). A calibration curve of absorbance vs. concentration was constructed using Beer's Law, A=εb c, where A is the measured absorbance, c is the molar concentration of analyte, b is the path length in cm, and ε is the molar absorptivity with units of liter per mole per centimeter (L·mol⁻¹·cm⁻¹), to determine the concentration of β-carotene in each mixing sample analyzed.

### Materials Preparation

### Example 1

### CIJ Mixing for In Situ Formation of PS-b-PEG Without β-Carotene

In a dry box under an argon atmosphere, 0.12 g (6.0×10⁻⁵ mol) of 2000 g/mol PS—COCl and 0.30 g (6.0×10⁻⁵ mol) of 5000 g/mol PEG-NH₂ were added to separate dry volumetric flasks. The flasks were removed from the dry box and the polymers were dissolved in 10 mL each of THF and water, respectively, to make a 4.2 wt-% solution at the inlet of mixing.

Mixing experiments were carried out by loading polymer solutions into separate 100 mL gas tight glass syringes (SGE, Inc.) and then attaching them to the syringe pump and mixing apparatus. Mixing occurred by impinging two streams into the mixing head at a flow rate of 77 mL/min or a jet velocity of 6.5 m/sec. The stream coming out of the mixer was directed into a glass jar containing 80 mL of distilled and filtered water to yield a final polymer concentration of 0.42 wt-% (6.0×10⁻⁴ M).

At the beginning of one mixing experiment, the stream was directed into a vial containing a 20 M excess of propylamine to quench the reaction and the remainder of the solution was directed into a glass jar containing 80 mL of distilled water as above.

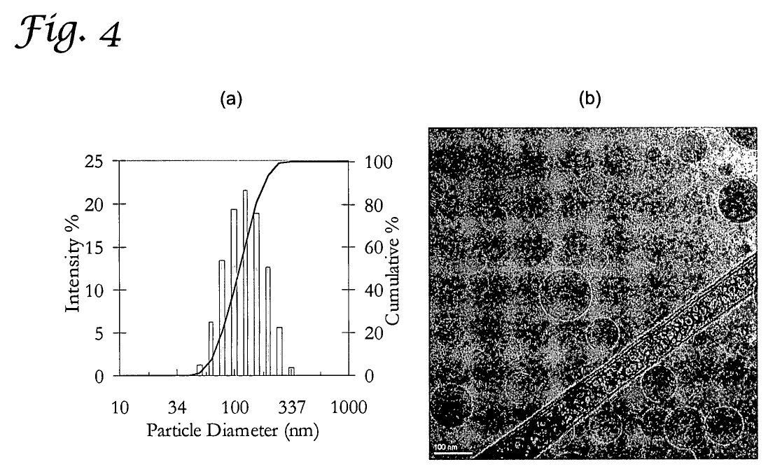

FIG. 4 shows the results from DLS and Cryo-TEM from reactive coupling. The average particle size by cumulant analysis and by REPES is 122 nm and 121 nm, respectively. Since the distribution is monodisperse and two methods of analysis are in agreement, all particle sizes determined by DLS were analyzed using the cumulant method (see, e.g., Koppel, D. E. J. Chem. Phys. 1972, 57, 4814-4820), and polydispersity was measured by the REPES method. This run was repeated two more times for a total of three runs. The average particle size by reactive coupling in THF/water through the mixer is 129±5 nm as determined by the cumulant method. Results from all three runs are summarized in Table 1. This average size seems to be in agreement with particles seen in the Cryo-TEM image. Particle sizes are larger than expected from the premade results due to the presence of unreacted polystyrene (approximately 50% unreacted PS present in this sample), which has an affinity for the core of particles. Approximately 25% of the PS—COCl sample used was converted to PS—COOH before reaction and approximately 25% more did not react with PEG-NH₂ before exiting the mixer. Thus, these particles are swollen slightly by unreacted PS. To avoid this, one should ensure that PS—COCl is 100% functional before reaction with PEG-NH₂. In addition, there is likely a higher amount of unreacted PS present because some of the PS—COCl did not form block copolymer. Based the GPC results at the outlet of the mixer, it was determined that approximately 50% of the PS added to the mixer did not form a block copolymer. Since there is now 50% unreacted PS rather than 25% in the premade case, it makes sense that the average particle size increased when moving from premade to reactively formed block copolymers. This experiment has proven that reactive coupling in a two solvent mixing stream is successful in forming block copolymer particles that is as effective as premaking the block copolymer.

TABLE 1
Experimental Results for in situ Formation
of PS-b-PEG without β-Carotene

| | Inlet Polymer | | | PDIₗ by |
|---|---|---|---|---|
| | Concentration | | D̅ₗ̅ (nm) | DLS |
| Run Number | wt-% | Re | by DLS | REPES |
| 1 | 4.2 | 1880 | 121 | 1.12 |
| 2 | 4.2 | 1880 | 122 | 1.12 |
| 3 | 4.2 | 1880 | 143 | 1.16 |

### Example 2

### CIJ Mixing for In Situ Formation of PS-b-PEG with β-Carotene

PS-b-PEG (Mn=2000-b-5000 g/mol) was prepared by reactively mixing stoichiometric amounts of PS—COCl or PS—COOH in THF with PEG-NH₂ in HPLC grade water at various concentrations (assuming PS—COCl and PEG-NH₂ were about 100% functional). β-carotene was dissolved in the PS containing THF solution before loading. The concentrations at mixing are summarized in Table 2. Mixing experiments were carried out by loading the 10 mL PS—COCl solution in THF into an 100 mL gas tight glass syringe, attaching the syringe to the syringe pump and impingement mixer. A second syringe was loaded with 10 mL of distilled water containing PEG-NH₂ and also attached to the mixing apparatus. Mixing occurred by impinging the two streams into the mixing head at a flow rate ranging from 20-77 mL/min. The stream coming out of the mixer (outlet fluid) was directed into a glass jar containing 80 mL of distilled and filtered water to yield a final concentration of product polymer of 1.8×10⁻⁶ M to 1.3×10⁻⁵ M (0.13 to 0.91 wt-%) assuming all functional polymer had formed a diblock copolymer. After mixing, the resulting solution was dialyzed against distilled and filtered water for 48 hours to remove the THF from the solution.

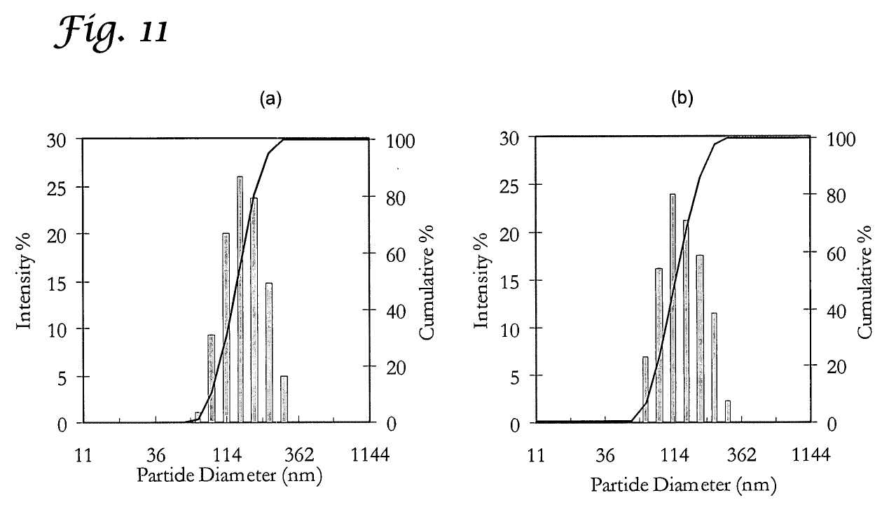

TABLE 2
Experimental Results for in situ Formation of PS-b-PEG with β-Carotene.

| | | | | | | | | | | | | | Intensity |
|---|---|---|---|---|---|---|---|---|---|---|---|---|---|
| | | | | Inlet | Outlet | Inlet | | | | | | | Particle |
| | | | | Polymer | Polymer | β- | Outlet | | D̅ₗ̅ | PDIₗ | | wt-% β- | Size |
| | | | PEG- | Concen- | Concen- | Caro- | β- | Reynolds | by | by | D̅ₗ̅ by | Carotene | Distribution |
| Run | PS—COOH | PS—COCl | NH₂ | tration | tration | tene | Carotene | Number | DLS | DLS | Coulter | Encap- | By DLS |
| Number | (g) | (g) | (g) | wt-% | wt-% | wt-% | wt-% | (Re) | (nm) | REPES | (nm) | sulated | REPES |
| 1 | — | 0.036 | 0.09 | 1.3 | 0.65 | 2.6 | 1.3 | 1880 | 163 | 1.19 | — | — | |
| 2 | — | 0.07 | 0.19 | 2.6 | 1.3 | 2.6 | 1.3 | 1880 | 170 | 1.42 | — | 82 | |
| 3 | — | 0.12 | 0.32 | 4.2 | 2.1 | 2.6 | 1.3 | 1880 | 201 | 1.07 | — | 81 | |
| 4 | — | 0.12 | 0.32 | 4.2 | 2.1 | 2.6 | 1.3 | 1880 | 148 | 1.12 | — | 80 | |
| 5 | — | 0.12 | 0.32 | 4.2 | 2.1 | 2.6 | 1.3 | 1880 | 150 | 1.08 | — | — | FIG. 11 (a) |
| 6 | — | 0.12 | 0.32 | 4.2 | 2.1 | 2.6 | 1.3 | 1880 | 144 | 1.10 | — | — | FIG. 11 (b) |
| 7 | — | 0.26 | 0.65 | 9.1 | 4.5 | 2.6 | 1.3 | 1880 | 177 | 1.14 | — | — | |
| 8 | — | 0.12 | 0.32 | 4.2 | 2.1 | 2.6 | 1.3 | 492 | 185 | 1.14 | — | — | |
| 9 | — | 0.12 | 0.32 | 4.2 | 2.1 | 2.6 | 1.3 | 955 | 162 | 1.06 | — | 77 | |
| 10 | — | 0.12 | 0.32 | 4.2 | 2.1 | 1.3 | 0.65 | 1880 | 143 | 1.07 | — | — | |
| 11 | — | 0.12 | 0.32 | 4.2 | 2.1 | 5.2 | 2.6 | 1880 | 157 | 1.09 | — | 55 | |
| 12 | 0.12 | — | 0.32 | 2.6 | 1.3 | 2.6 | 1.3 | 1880 | — | — | ˜500 | — | — |

The results of all mixing experiments discussed throughout this series of experiments are summarized in Table 2. Note that the 4.2 wt-% block copolymer and 2.6 wt-% β-carotene sample was run a total of four times yielding an average particle size of 161±27 nm if all four runs are taken into consideration or 147±3 nm if the largest average is considered an outlier. Since the standard deviation of both averages suggests that this first run lies far from the others, beyond one standard deviation for both, then, statistically, this run should not be considered. Therefore, the average particle size of 147 nm for this run will be considered. There is a standard deviation of only 3 nm for these runs, which means there is high reproducibility between runs, and so one run should be enough to explore the effects of polymer concentration, jet velocity, and β-carotene concentration on particle size.

An PDI_{I} was calculated for each run using the results from REPES as a measure of the distribution width. Results of PDI_{I} are included in Table 2. Breadths of distributions are narrow, as all of distributions are approximately 1.1. A PDI close to 1 would indicate a mono disperse set of particle sizes, that has very little variation in size.

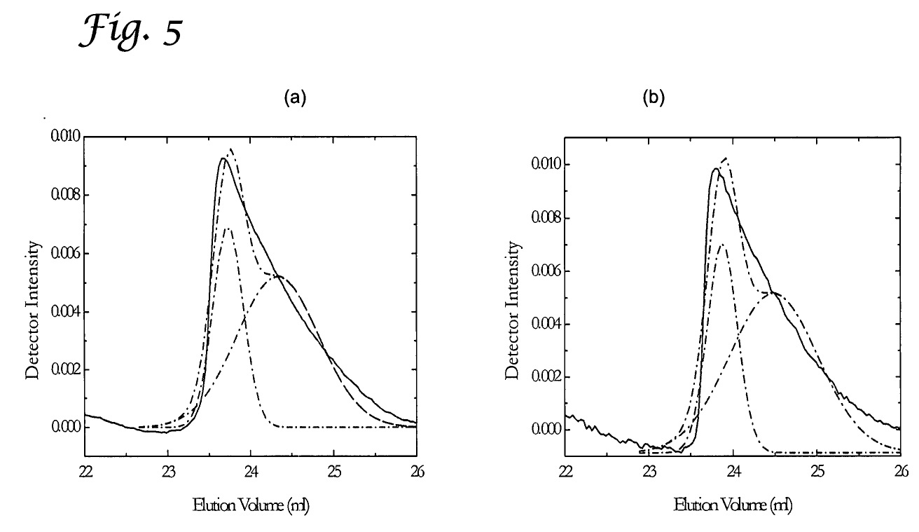

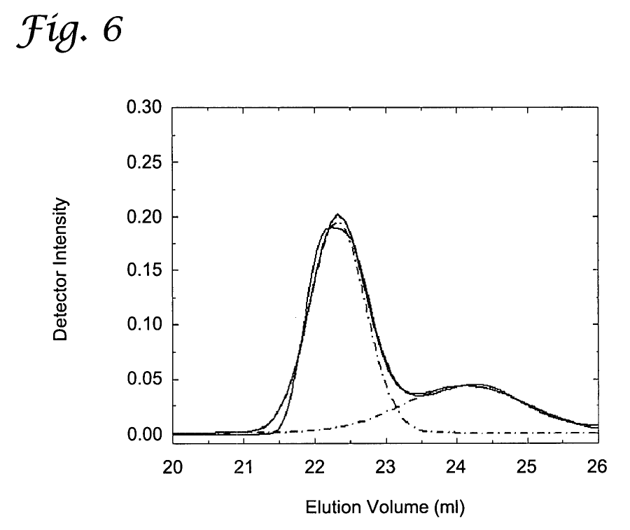

FIG. 5 gives GPC plots for two of runs from this study. The solid line on each plot is the output of the UV detector and broken lines are Gaussian deconvolution plots of the UV signal. With correction for PS functionality of 75% PS—COCl, as determined above, conversion at the outlet of the mixer for these runs was approximately 68%, which is comparable to the result of 70% before the addition of β-carotene to the reaction scheme from FIG. 6. The GPC plots are similar for both runs and to the run without β-carotene suggesting that block copolymer has formed in the presence of β-carotene and this chemical does not interfere with the formation of block copolymer. Note that the peak elution for the block copolymer is 24.84 mL, a drift of 0.5 ppm from FIG. 6. Slight drifts in actual elution volume are common in GPC, especially after a solvent change, which is why a new calibration is necessary each time GPC is used. These results also indicate that approximately 50% of the PS does not form block copolymer in a two solvent stream.

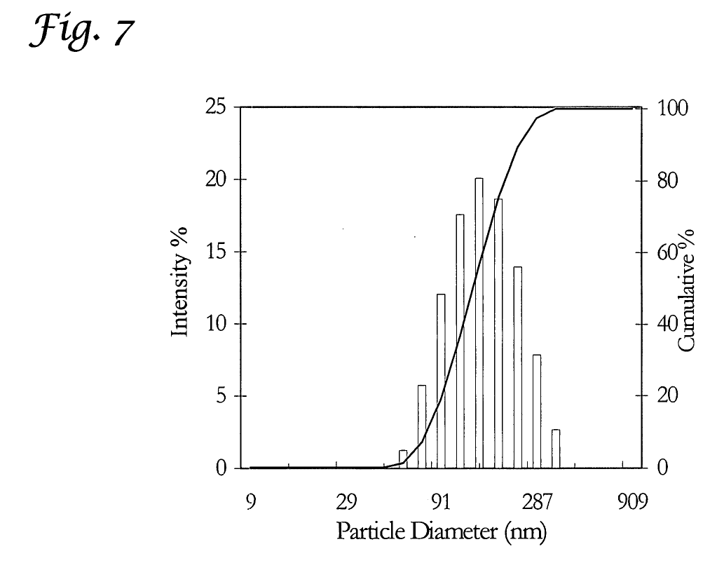

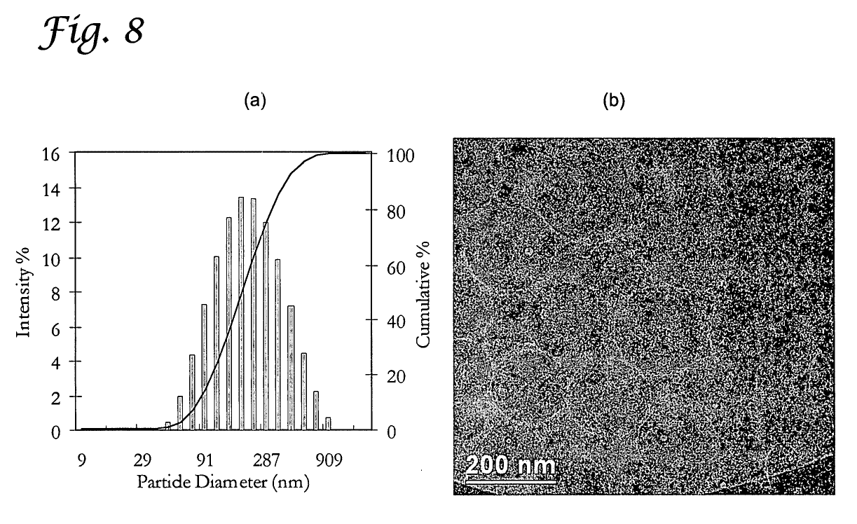

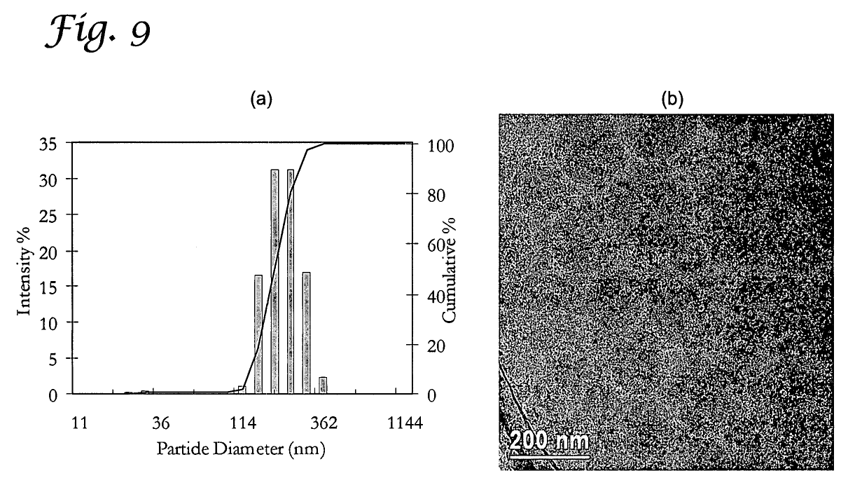

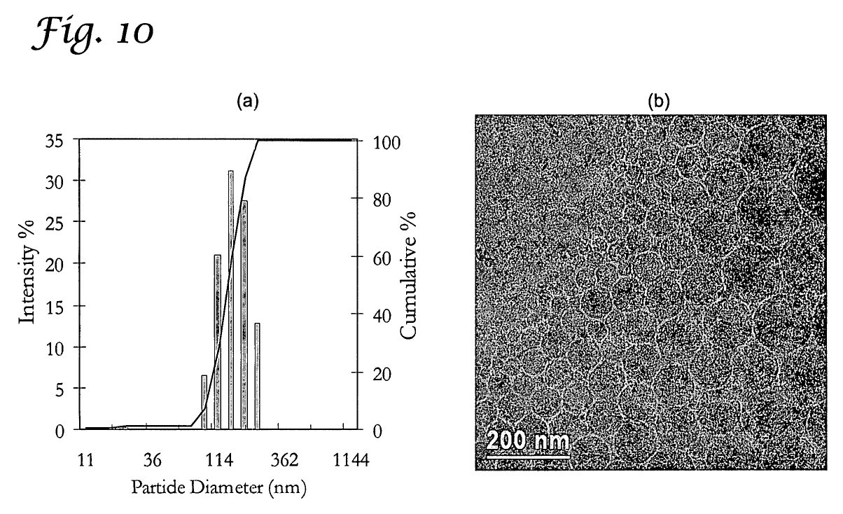

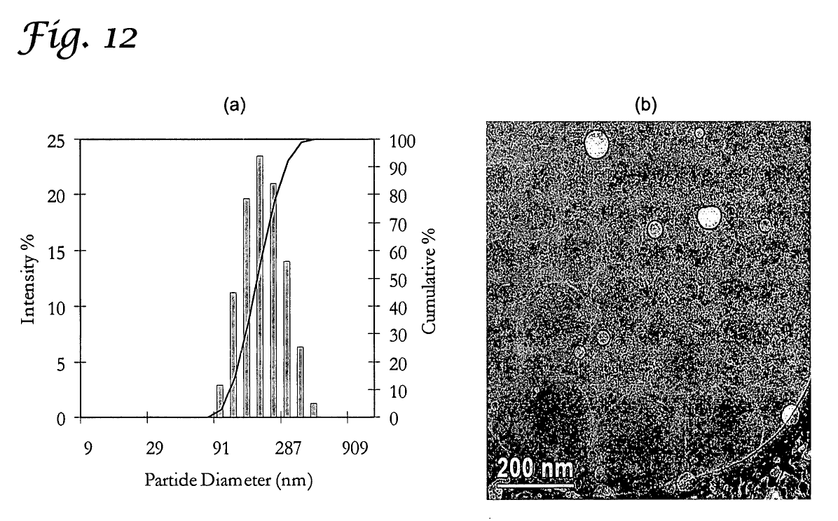

DLS was run for each sample to determine particle size for all four polymer concentrations and Cryo-TEM was run determine the shape of block copolymer particles for the concentrations of block copolymer ranging from 2.6-9.1 wt-% at a constant concentration of β-carotene. The DLS results by cumulant analysis for all experiments are summarized above in Table 2. In addition, results for DLS by REPES as well as Cryo-TEM images for each concentration are summarized in FIGS. 7-12, respectively. Results from DLS suggest that the average particle size remains relatively consistent regardless of the polymer concentration. The narrowest distribution is for 4.2 wt % block copolymer, which is a molar concentration equivalent to Johnson's work with a 1000-b-3000 g/mol analogous block copolymer (see, e.g., Johnson, B. K. Ph.D. Dissertation Flash Nanoprecipitation of Organic Actives via Confined Micromixing and Block Copolymer Stabilization, 2003, Department of Chemical Engineering, Princeton University, and Jakes, J. Collect. Czech. Chem. Comm. 1995, 60, 1791-1797). Johnson was unable to make particles with β-carotene encapsulated under 300 nm in diameter without decreasing initial concentrations of block copolymers and β-carotene to less than or equal to 0.52 wt-%. However, REPES and Cryo-TEM analysis suggest that the distribution about the mean is greater at low and high polymer concentration. The narrowest distribution is for 4.2 wt-% block copolymer. Here, by increasing the molecular weight and by forming the block copolymer through reactively coupling in situ, an average particle size less than 200 nm was achieved.

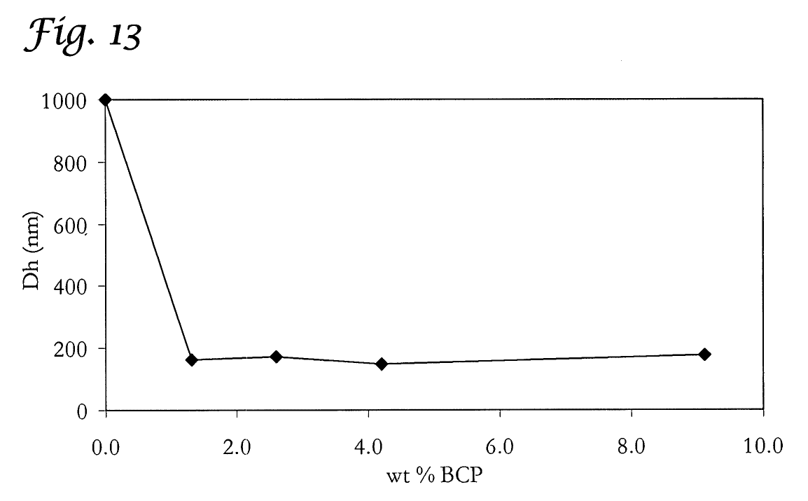

FIG. 13 is a plot of intensity average particle sizes, D̅_̅{̅I̅}̅, as a function of the block copolymer concentration. This plot suggests that there is little or no effect on the particle size when the β-carotene concentration is held constant and the concentration of block copolymer is varied. However, a complete lack of block copolymer creates micron-sized particles. Therefore, the in situ formation of block copolymer particles is effective in colloidal stabilization of β-carotene in the nanometer size range for concentrations of polymer and β-carotene studied.

### Example 3

### Analogous Experiment on In Situ Formation of PS-b-PEG with β-Carotene

As a control experiment to prove block copolymer formation and encapsulation of β-carotene in block copolymer particles, an analogous experiment was carried out using PS—COOH in place of PS—COCl. Acids do not react with amines in the time scale in which acid chlorides react with amines (see, e.g., Orr et al. Polymer 2001, 42, 8171-8178), so this study should demonstrate the role of the reaction between the acid chloride and amine on block copolymer particle formation with β-carotene. The sample could not be analyzed by DLS because they had no signal after filtering though a 0.42 μm filter, suggesting that all of particle sizes were greater than 420 nm. Since all of samples made with PS—COCl and PEG-NH₂ were less than 300 nm by DLS, there is further evidence that particles illustrated in FIGS. 7-10 are indeed block copolymer particles with β-carotene encapsulated inside.

To determine particle sizes of the sample made with PS—COOH and PEG-NH₂, a Coulter LS230 that uses Mie theory was used to determine exactly how large particles were. Table 2 summarizes results of mixing PS—COOH with PEG-NH₂. This method of particle size analysis is most valid for large particles (greater than 500 nm) even though the company claims a valid detection limit of 40 nm. Below 500 nm, Mie theory loses validity and so results become less reliable below this size (see, e.g., Lyu, S. Ph.D. Dissertation Particle Coalescence in Polymer Blends during Shearing, 2000, Department of Chemical Engineering and Materials Science and Engineering, University of Minnesota). Particle size analysis using Mie theory was attempted for PS—COCl+PEG-NH₂ samples, but results were not consistent with DLS and Cryo-TEM.

### Example 4

### Effect of Jet Velocity on Particle Sizes

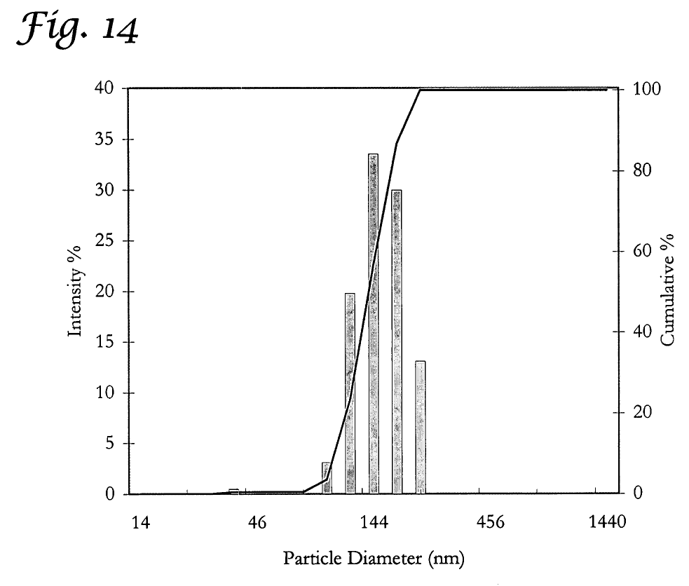

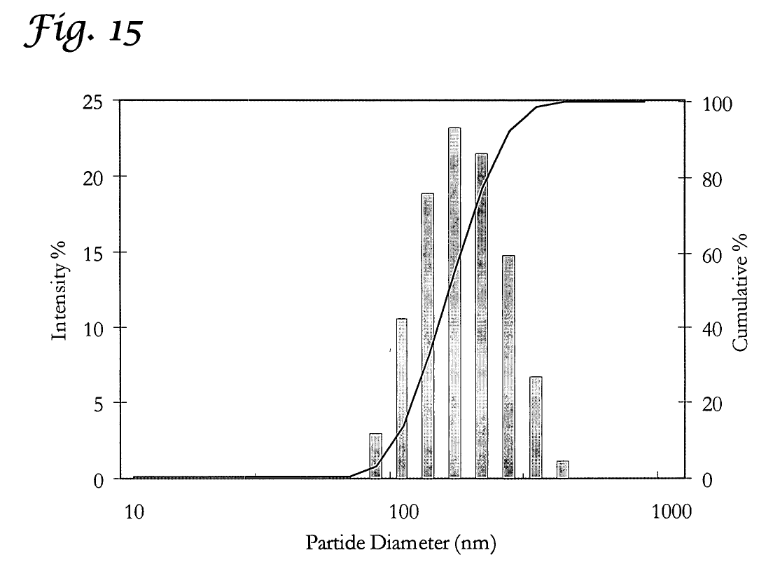

To determine the role of the jet velocity on the particle size, experiments were run at three different jet velocities, 6.5 m/sec (a flow rate of 77 mL/min and Re=1880), 3.3 m/sec (a flow rate of 39 mL/min and Re=955) and 1.7 m/sec (a flow rate of 20 mL/min and Re=492) keeping the block copolymer and β-carotene concentrations constant. The Reynolds number (Re) (Re=ρdv/η where ρ is the density, d is the jet diameter, v is the jet velocity and η is the viscosity of the solvent) was calculated using the viscosity for a 1:1 mixture of THF:water (see, e.g., Johnson, B. K. Ph.D. Dissertation Flash Nanoprecipitation of Organic Actives via Confined Micromixing and Block Copolymer Stabilization, 2003, Department of Chemical Engineering, Princeton University). A jet velocity of 6.5 m/sec was used throughout this study, and 1.7 m/sec should be near or just below the breakpoint for PS-b-PEG (see, e.g., Johnson, B. K. Ph.D. Dissertation Flash Nanoprecipitation of Organic Actives via Confined Micromixing and Block Copolymer Stabilization, 2003, Department of Chemical Engineering, Princeton University). Results from DLS using cumulant analysis are given in Table 2 along with the PDI_{I} calculated for each run calculated from REPES results. Results from REPES and Cryo-TEM are also presented in FIGS. 14-15. The cumulant analysis results suggest that particle size increases with decreased jet velocity, but REPES and Cryo-TEM suggest that there is no noticeable change in particle size with jet velocity for the two higher jet velocities. The third jet velocity, 1.7 m/sec shows a significant increase in particle size. Also, in comparison to the results from the previous section there is no significant change in particle size for the two higher jet velocities. The break point is defined as the jet velocity in which an increase in jet velocity has no effect on the resulting particle size, which was determined to be 2.8 m/sec for PS-b-PEG (see, e.g., Johnson, B. K. Ph.D. Dissertation Flash Nanoprecipitation of Organic Actives via Confined Micromixing and Block Copolymer Stabilization, 2003, Department of Chemical Engineering, Princeton University; Johnson et al. AIChE J. 2003, 49, 2264-2282; and Johnson et al. Phys. Rev. Lett. 2003, 91, 1183021-1183024).

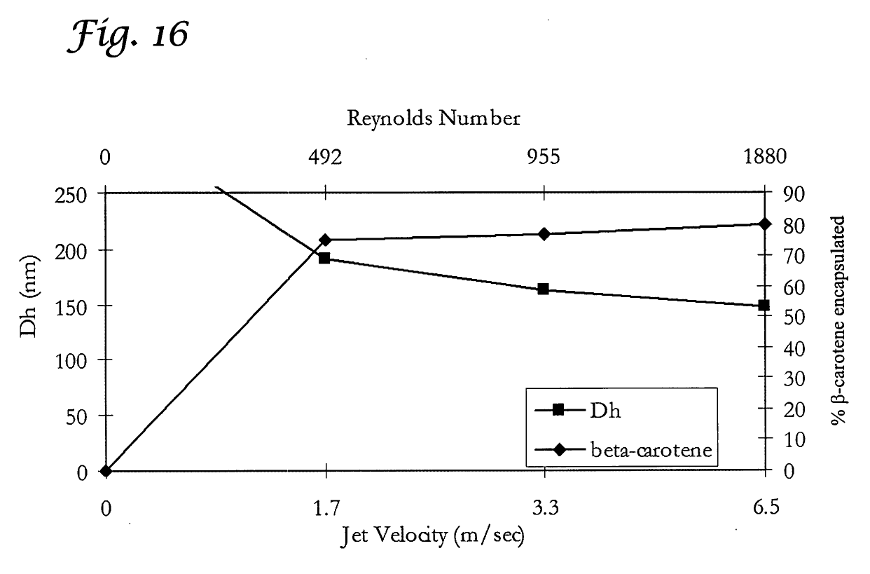

A plot illustrating the relationship between jet velocity and particle size is given in FIG. 16. This trend line suggests that at high velocity, there is no effect of jet velocity on particle size; however, it appears as though below 2.0 m/sec the particle size increases dramatically.

### Example 5

### Effect of β-Carotene on Encapsulation

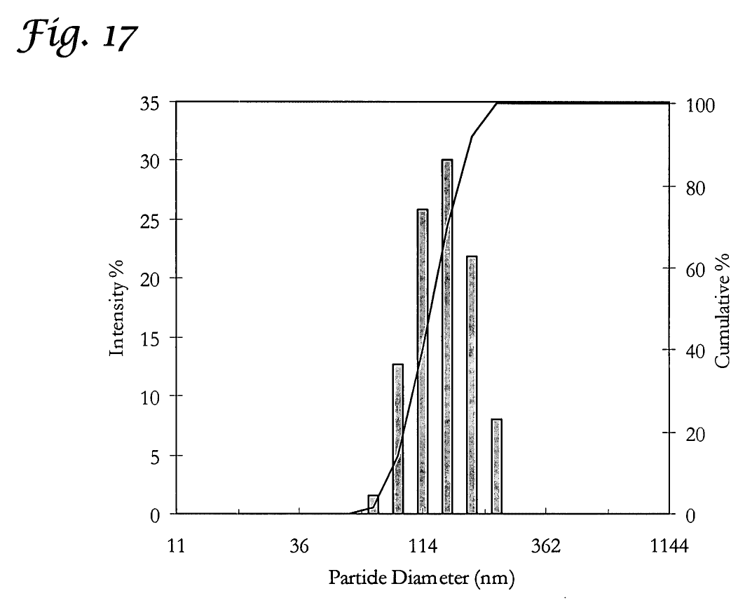

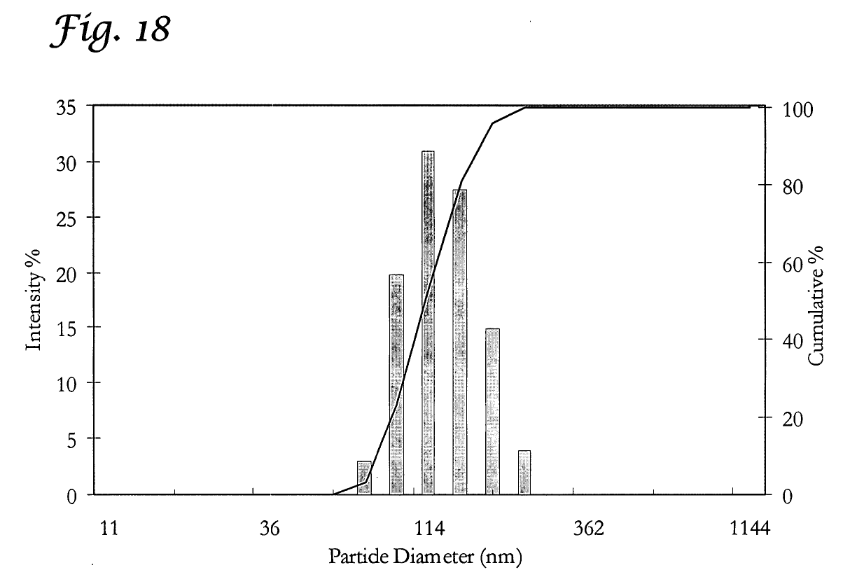

The purpose of this series of experiments was to determine the effect of increased β-carotene concentration on the average particle size. Results from DLS cumulant analysis are summarized in Table 2. REPES results for decreased β-carotene concentration are summarized in FIG. 17. REPES results for increased β-carotene concentration are summarized in FIG. 18. Results from DLS suggest that there is no significant change in particle size for either sample. However, it should also be noted that there was increased precipitation of unencapsulated β-carotene at the bottom of the reaction vessel by visual inspection within one hour of reaction for the increased concentration of β-carotene and not for the lower concentrations. This observation was confirmed by UV-vis, which found only 55% of the β-carotene was encapsulated when 5.2 wt-% β-carotene was added to the mixing stream. This suggests that one cannot increase the β-carotene concentration for this system, it is already are at the saturation limit with 2.6 wt-% β-carotene. By increasing the concentration, the supersaturation has increased. So when β-carotene comes into contact with water, β-carotene particles nucleate and grow faster than PS-b-PEG can encapsulate them and arrest growth.

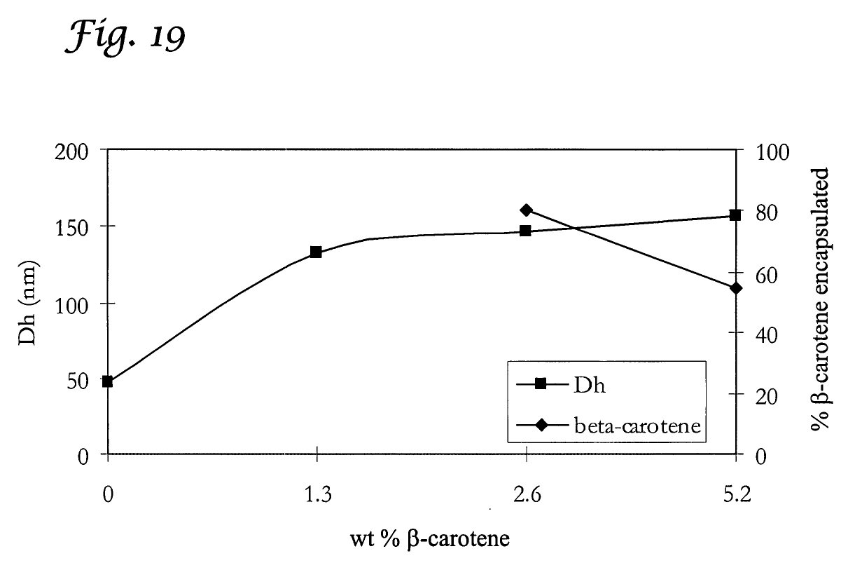

FIG. 19 is a plot of intensity average particle sizes as a function of β-carotene concentration. This trend line suggests that the upper concentration limit for β-carotene in a 4.2 wt-% solution of PS-b-PEG is 2.5 wt-% as there is no significant increase in particle size with an increase in β-carotene concentration and the sedimentation of β-carotene has increased for this sample.

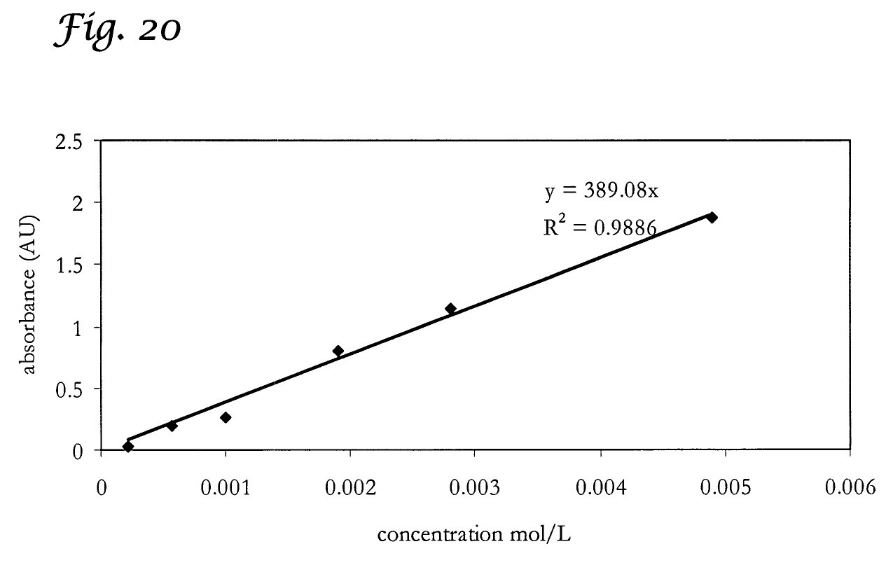

UV-vis was used to determine how much β-carotene was encapsulated in a few of the samples made in this set of experiments. It was assumed that all of premade block copolymers used in this study were able to encapsulate 100% of the β-carotene as evidenced by a lack of opacity in the solution and a lack of precipitation formation over an extended period of time (6 months or more). The β-carotene encapsulated in the reactively formed block copolymer particles as determined by UV-vis are summarized in Table 2. All of samples investigated were analyzed using the calibration curve for absorbance vs. concentration in FIG. 20. The slope of this line is used to determine the concentration of β-carotene in the solution prepared from the precipitate in the mixing samples. The concentration is then converted to the amount of precipitate present by accounting for the volume of the prepared solution. For all of samples where 2.6 wt-% β-carotene was used, approximately 80% of the β-carotene was encapsulated by the particles. However, for the 5.2 wt-% β-carotene sample, only 55% was encapsulated. Since there was so much more β-carotene that precipitated from the higher concentration, it is logical that there was not a significant increase in particle size when the concentration of β-carotene increased. The higher concentration of β-carotene caused more rapid nucleation and growth, so less could be encapsulated before aggregates became too large for particles to surround them and arrest nucleation and growth.

It is also worth noting that UV-vis was used to determine how much β-carotene was encapsulated by measuring the concentration of the precipitate for several reasons. First, the method easily distinguished between free PS that may have precipitated and free β-carotene because the wavelength of maximum absorbance for PS in THF is 254 nm, the UV-vis region, whereas the wavelength of maximum absorbance for β-carotene is 485 nm, the blue area of the visible region. Since these two wavelengths are so far apart in the spectrum, there will be no overlap in the absorbance spectra for these two components. Secondly, since the exact percentage of PS or β-carotene in the precipitate was unknown, there would have been no way to determine how much β-carotene was encapsulated by direct gravimetric analysis. Finally, the precipitate was weighed because there was presumably less β-carotene present there than in solution, and drying particles and redissolving them in would have been a more arduous task than simply drying the precipitate.

### Example 6

### Synthesis of PCL-COCl

Capric acid (0.91 g, 6.3 mmol), ε-caprolactone (14.4 g, 126 mmol) and camphor sulfonic acid (CSA, 5.0 mg) were added to a 50 mL culture tube. The mixture was purged with nitrogen for 10-15 min. The tube was put in a pre-heated sand bath at 230° C. for 24 hours (h). The reaction was quenched by cooling the system to ambient temperature. ¹H NMR spectroscopy of the crude mixture indicated that the conversion of ε-caprolactone was 95%. The crude product was fractionated into four fractions [7100 g/mol (PDI=1.24, 4.7 g); 5100 g/mol (PDI=1.21, 3.6 g); 3600 g/mol (PDI=1.21, 1.8 g); 2400 g/mol (PDI=1.14, 1.5 g)] by dissolving the mixture in THF, followed by slow sequential addition of methanol.

PCL-COOH (Mₙ=2400 g/mol, 1.45 g, 0.60 mmol) was dissolved in 10 mL of dry dichloromethane in a 25 mL flask. About 10 μL of DMF was added via a WIRETROL micropipette to the flask, and followed by the addition of oxalyl chloride (0.54 mL, 6.0 mmol) via a syringe. The reaction mixture was stirred at room temperature for 12 h. Dichloromethane and excess oxalyl chloride were removed under vacuum to afford PCL-COCl.

### Example 7

### Verification of Formation of Block Copolymer PCL-b-PEG Via Coupling Reaction

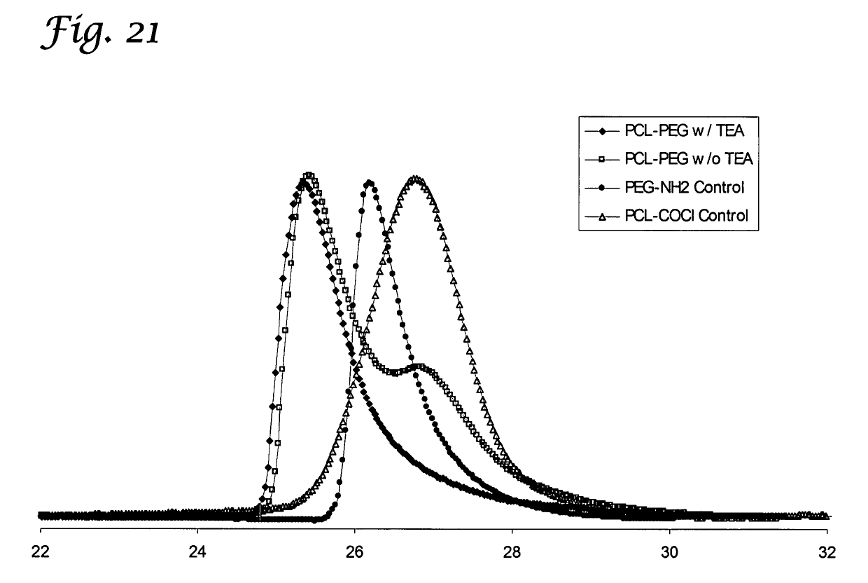

A coupling reaction before CIJ mixing was employed to verify formation of the block copolymer PCL-b-PEG. Typical synthesis procedures and conversion measurement were as follows. a) A 0.0032 g (1.5×10⁻⁶ mol) of PCL-COCl (Mₙ=2400 g/mol) was weighted in a glove box, and a 0.0090 g (1.5×10⁻⁶ mol) of PEG-NH₂ (Mₙ=6000 g/mol) in air (molar ratio 1:1). These were mixed together into 4 mL of THF in a dry GPC vial; b) Repeated step a) and added 5 μL (6×10⁻⁵ mol) of triethylamine (TEA) into the second mixture; c) Above two samples were let sit on bench for 30 minutes (min) before quenching and GPC loading; d) Weighted two control samples, 0.0030 g (1.25×10⁻⁶ mol) of PCL-COCl (Mₙ=2400 g/mol) and 0.0051 g of (8.5×10⁻⁷ mol) PEG-NH₂ (Mₙ=6000 g/mol), and dissolved them into 4.0 mL of THF in a dry GPC vial, separately. e) Added two drops of phenyl isocyanate C₆H₅NCO into above solutions to quench free —NH₂ groups, which could falsely delay GPC elution; f) Loaded above two premade samples, two controls and two sets of polystyrene standards for a GPC measurement. The GPC trace in FIG. 21 showed a high coupling conversion of at least 90% in the presence of TEA. However, a coupling conversion was below 50%, if TEA was not added. Therefore, TEA was necessary to add for achieving a high yield of the block copolymer.

### Example 8

### CIJ Mixing for In Situ Formation of PCL-b-PEG Without β-Carotene

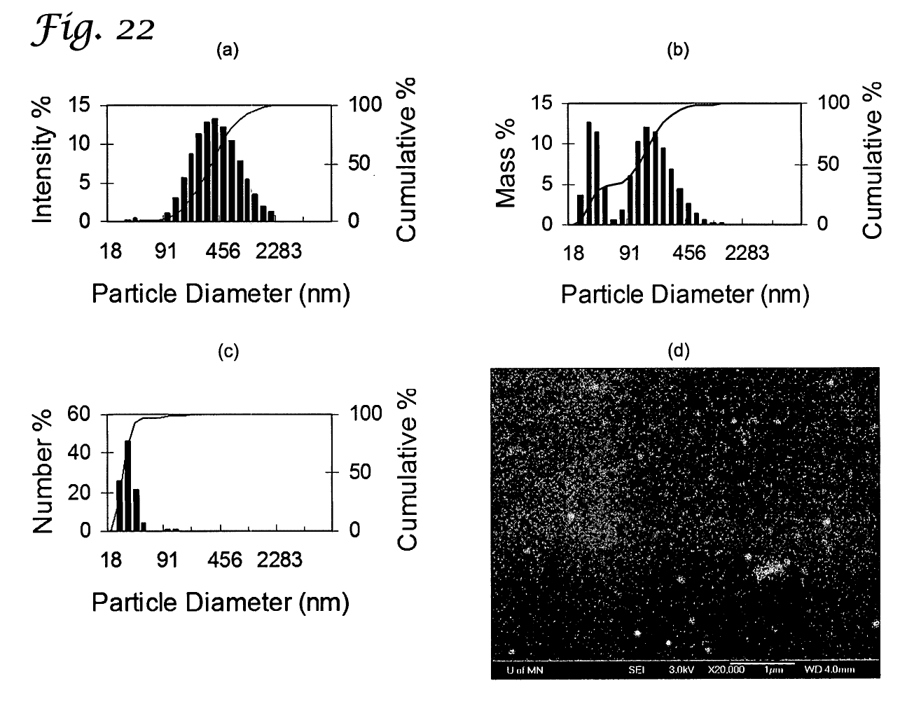

A 0.108 g (3×10⁻⁵ mol) of PCL-COCl (Mₙ=3600 g/mol) was weighted in a glove box and dissolved in 5.0 mL of THF. A 20 μL (2.5×10⁻⁴ mol) of TEA was then added into this solution. A 0.180 g (3×10⁻⁵ mol) of PEG-NH₂ (Mₙ=6000 g/mol) was weighted in air and dissolved in 5.0 mL of H₂O. Two solutions were loaded respectively into two 100 mL gas tight glass syringes and attached to the CIJ mixing apparatus. Mixing was performed at a flow rate of 72 mL/min and Reynolds number (Re) of 1780. The 10 mL of the fluid coming out of the outlet tubing (polymers 2.88 wt-%) was diluted by 40.0 mL of HPLC grade water in ajar to yield a final concentration of polymers of 0.576 wt-% (6.0×10⁻⁴ mol/L). Experimental results are shown in Table 3 and FIG. 22.

### Example 9

### CIJ Mixing for In Situ Formation of PCL-b-PEG with β-Carotene

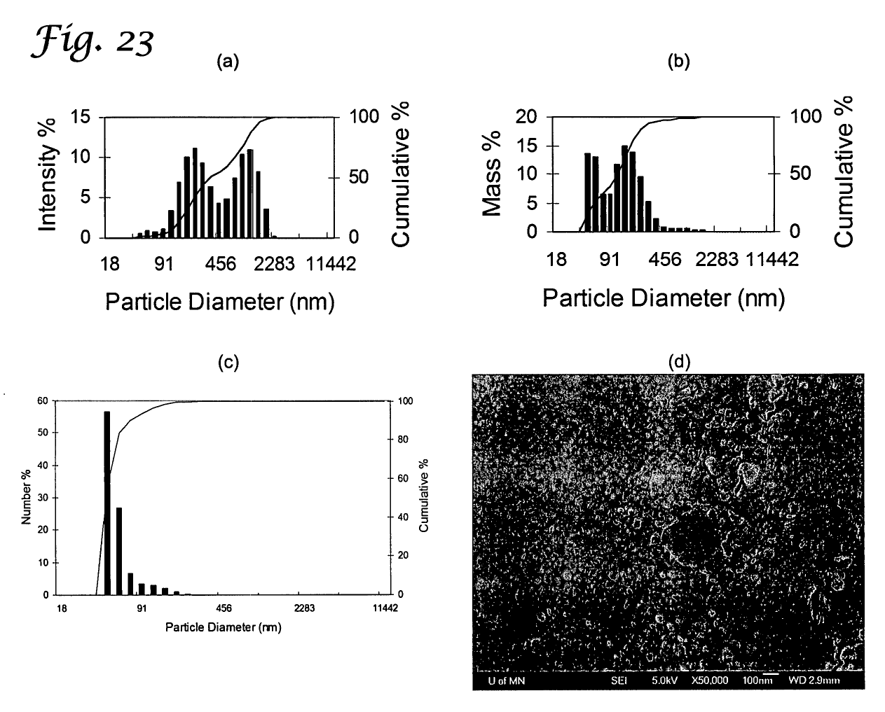

A 0.108 g (3×10⁻⁵ mol) of PCL-COCl (Mₙ=3600 g/mol) was weighted in a glove box and dissolved with a 0.130 g of β-carotene in 5.0 mL of THF. A 20 μL (2.5×10⁻⁴ mol) of TEA was then added into this solution. A 0.180 g (3×10⁻⁵ mol) of PEG-NH₂ (Mₙ=6000 g/mol) was weighted in air and dissolved in 5.0 mL of H₂O. Two solutions were loaded respectively into two 100 mL gas tight glass syringes and attached to the CIJ mixing apparatus. Mixing was performed at a flow rate of 72 mL/min and Reynolds number (Re) of 1780. The 10 mL of the fluid coming out of the outlet tubing (polymers 2.88 wt-%, β-carotene 0.65 wt-%) was diluted by 40.0 mL of HPLC grade water in ajar to yield a final concentration of polymers of 0.576 wt-% (6.0×10⁻⁴ mol/L) with β-carotene of 0.130 wt-%. Experimental results are shown in Table 3 and FIG. 23.

TABLE 3
Experimental Results for Mixing in situ Formed PCL-b-PEG with and without β-Carotene

| | | | | | Outlet | Outlet β- | | | | |
|---|---|---|---|---|---|---|---|---|---|---|
| | | PEG-NH₂ | PEG-b-PEG | | Polymer | Carotene | | D̅ₗ̅/ D̅_̅{̅w̅}̅/ | | Intensity/Mass/ |
| | PCL—COCl | (6 | (3.6-b-6 | β- | Concen- | Concen- | Reynolds | D̅ₙ̅ (nm) | PDIₗ/PDI_{w}/ | Number Size |
| Run | (3.6 kg/mol) | kg/mol) | kg/mol) | Carotene | tration | tration | Number | by DLS | PDIₙ by DLS | Distribution by |
| Number | (g) | (g) | (g) | (g) | wt-% | wt-% | (Re) | REPES | REPES | DLS REPES |
| 1 | 0.108 | 0.180 | — | — | 2.88 | — | 1780 | 454/153/32 | 1.53/1.84/1.25 | |
| 2 | 0.108 | 0.180 | — | 0.065 | 2.88 | 0.65 | 1780 | 614/154/58 | 1.63/1.91/1.20 | |

The complete disclosure of all patents, patent applications, and publications, and electronically available material (e.g., GenBank amino acid and nucleotide sequence submissions; and protein data bank (pdb) submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of producing nanoparticles, the method comprising:
providing a first reactant mixture comprising a first solvent stream and one or more polymeric reactants, wherein the first solvent stream comprises one or more solvents;
providing a second reactant mixture comprising a second solvent stream and one or more polymeric reactants, wherein the second solvent stream comprises one or more solvents; and
combining the reactant mixtures and flash precipitating under conditions to react the polymeric reactants and form precipitated nanoparticles comprising a block and/or graft copolymer.

2. The method of claim 1 wherein flash precipitating the nanoparticles is performed in a centripetal mixer, a continuous flash mixer, or a batch flash mixer.

3. The method of claim 2 wherein the mixing velocity has a Reynold's number of at least 100.

4. The method of claim 1 wherein the concentration of the polymeric reactant in each reactant mixture is at least 0.1 wt-%.

5. The method of claim 1 wherein the nanoparticles have a polydispersity index of less than 2.

6. The method of claim 1 wherein the nanoparticles have an average particle size of less than 1000 nm.

7. The method of claim 1 wherein at least one of the reactant mixtures comprises a polymeric reactant selected from functionalized polystyrenes, functionalized polycaprolactones, polyacrylates, functionalized polyethyleneglycols, polyglutamic acids, hyaluronic acids, functionalized polyvinylpyrrolidones, polylysines, polyspermines, polyarginines, alginic acids, functionalized polylactides, polyethyleneimines, polyacrylic acids, polyiminocarboxylates, and mixtures thereof.

8. The method of claim 7 wherein at least one of the reactant mixtures comprises a polymeric reactant selected from functionalized polystyrenes, functionalized polycaprolactones, and mixtures thereof.

9. The method of claim 7 wherein at least one of the reactant mixtures comprises a polymeric reactant selected from functionalized polyethylene glycols and mixtures thereof.

10. The method of claim 1 wherein at least one of the reactant mixtures comprises one or more polymeric reactants functionalized with an acid chloride group, an isocyanate group, an anhydride group, an epoxide group, a ketone group, an aldehyde group, an enone group, enoate group, an alkyne group, another electrophilic group, or mixtures thereof.

11. The method of claim 1 wherein at least one of the reactant mixtures comprises one or more polymeric reactants functionalized with an amino group, a hydroxyl group, a carboxylic acid group, a hydrazine group, a hydroxylamino group, a thiol group, an aromatic group, a heteroaromatic group, another nucleophilic group, or mixtures thereof.

12. The method of claim 1 further comprising providing one or more additional solvent streams comprising one or more solvents.

13. The method of claim 1 further comprising providing a third reactant mixture comprising a third solvent stream and one or more polymeric reactants, wherein the third solvent stream comprises one or more solvents.

14. The method of claim 1 further comprising a step of removing the solvents from the resultant mixture comprising the nanoparticles.

15. The method of claim 14 wherein the solvents are removed by a process selected from the group consisting of filtration, distillation, evaporation, expansion, spray drying, lyophilization, centrifugation, extraction, and combinations thereof.

16. The method of claim 1 further comprising providing one or more additive target molecules.

17. The method of claim 16 wherein providing one or more additive target molecules comprises providing the one or more additive target molecules in one or more of the reactant mixtures.

18. The method of claim 16 wherein providing one or more additive target molecule comprises providing the one or more additive target molecules in one or more solvent streams.

19. The method of claim 16 wherein the block and/or graft copolymer and the one or more additive target molecules are present in the nanoparticles in a ratio of at least 1:20 by weight.

20. The method of claim 16 wherein the block and/or graft copolymer and the one or more additive target molecules are present in the nanoparticles in a ratio of no greater than 99:1 by weight.

21. The method of claim 16 wherein at least one additive target molecule is selected from the group consisting of pharmaceutical actives and pharmaceutical precursor compounds.

22. The method of claim 16 wherein at least one additive target molecule is selected from the group consisting of cyclosporins, immunoactive agents, analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immuriological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, xanthines, antioxidants, preservatives, vitamins, nutrients, and combinations thereof.

23. The method of claim 16 wherein at least one additive target molecule is a peptide.

24. The method of claim 16 wherein at least one additive target molecule is selected from the group consisting of biocides, pesticides, herbicides, fungicides, insecticides, and combinations thereof.

25. The method of claim 16 wherein at least one additive target molecule is selected from the group consisting of cosmetic products, dyes, salts, biological markers, biological imaging agents, ink pigments, magnetic particles, radiopaque materials, and combinations thereof.

26. The method of claim 16 further comprising a step of removing the solvents from the resultant mixture comprising the nanoparticles.

27. The method of claim 26 wherein the solvents are removed by a process selected from the group consisting of filtration, distillation, evaporation, expansion, spray drying, lyophilization, centrifugation, extraction, and combinations thereof.

28. The method of claim 1 further comprising providing one or more supplemental additives.

29. Nanoparticles prepared according to the process of claim 1.

30. Nanoparticles prepared according to the process of claim 16.
